(19) United States
(12) Patent Application Publication
(10) Pub. No.: US 2020/0368174 A1
(43) Pub. Date: Nov. 26, 2020

(54) PARTICLES FOR TARGETED DELIVERY OF ACTIVE AGENTS INTO ADIPOSE STROMAL CELLS

(71) Applicants: Texas Tech University System, Lubbock, TX (US); University of Tennessee research Foundation, Knoxville, TN (US)

(72) Inventors: Shu Wang, Lubbock, TX (US); Ling Zhao, Knoxville, TN (US); Zhaoyang Fan, Lubbock, TX (US); Yujiao Zu, Lubbock, TX (US)

(73) Assignees: Texas Tech University System, Lubbock, TX (US); University of Tennessee research Foundation, Knoxville, TN (US)

(21) Appl. No.: 16/966,310

(22) PCT Filed: Feb. 21, 2019
(86) PCT No.: PCT/US2019/019036

§ 371 Date: Jul. 30, 2020

Related U.S. Application Data
(60) Provisional application No. 62/633,300, filed on Feb. 21, 2018.

(51) Int. Cl. A61K 9/51; A61K 31/05; A61K 47/69; A61K 47/62

(52) U.S. Cl. CPC A61K 9/5123; A61K 47/62; A61K 47/6929; A61K 31/05

(57) ABSTRACT

Embodiments of the present disclosure pertain to delivery agents for delivering one or more active agents to desired cells (e.g., adipose stromal cells). The delivery agents generally include: (1) a particle; (2) one or more active agents carried by the particle; and (3) a targeting agent associated with the particle, where the targeting agent directs the delivery agent to the desired cells. Additional embodiments of the present disclosure pertain to methods for delivering active agents to adipose stromal cells through the use of the aforementioned delivery agents. In some embodiments, the methods include a step of associating the adipose stromal cells with the delivery agent such that the associating results in the delivery of the active agents into the adipose stromal cells. The associating can occur by administering the delivery agent to a subject for the treatment or prevention of obesity and related disorder or diseases in the subject.

### CROSS-REFERENCE TO RELATED APPLICATIONS

This application claims priority to U.S. Provisional Patent Application No. 62/633,300, filed on Feb. 21, 2018. The entirety of the aforementioned application is incorporated herein by reference.

### STATEMENT REGARDING FEDERALLY SPONSORED RESEARCH

This invention was made with government support under Grant No. R15AT008733, awarded by the National Institutes of Health (NIH). The government has certain rights in the invention.

### BACKGROUND

Current compositions and methods of delivering active agents into adipose stromal cells have numerous limitations, including limited solubility, limited stability, limited bioactivities, and limited ability to reach desired adipose stromal cells. Various embodiments of the present disclosure address the aforementioned limitations.

### SUMMARY

In some embodiments, the present disclosure pertains to delivery agents for delivering one or more active agents to desired cells, such as adipose stromal cells. The delivery agents generally include: (1) a particle; (2) one or more active agents carried by the particle; and (3) a targeting agent associated with the particle, where the targeting agent directs the delivery agents to the desired cells (e.g., adipose stromal cells).

In additional embodiments, the present disclosure pertains to methods for delivering one or more active agents to adipose stromal cells through the use of the aforementioned delivery agents. In some embodiments, the methods of the present disclosure include a step of associating the adipose stromal cells with the delivery agents such that the associating results in the delivery of the one or more active agents into the adipose stromal cells. In some embodiments, a single type of particle that contains one or more active agents is utilized. In some embodiments, two or more different types of particles that each contain one or more of the same or different active agents are utilized.

In some embodiments, the associating occurs by administering the delivery agent to a subject. In some embodiments, the delivery agent is then used to treat or prevent obesity in the subject. In some embodiments, the delivery agent is used to treat or prevent a disorder or a disease in a subject. In some embodiments, the disorder or the disease is associated with obesity. In some embodiments, the disorder or disease can include, without limitation, metabolic syndromes, diabetes, type 2 diabetes, cardiovascular diseases, hypertension, coronary heart diseases, insulin resistance, dyslipidemia, cancer, osteoarthritis, rheumatoid arthritis, aging, wrinkles, alopecia, liver failure, multiple sclerosis, obesity, and combinations thereof.

### DESCRIPTION OF THE DRAWINGS

FIG. 1A provides an illustration of a delivery agent for delivering one or more active agents to adipose stromal cells.

FIG. 1B provides an example of a resveratrol (RES) delivery agent that is in the form of a nanoparticle (RES-NPs). The RES-NPs in this example include adipose stromal cell (ASC)-targeted peptides (i.e., the 1,2-distearoyl-sn-glycero-3-phosphoethanolamine-N-[(polyethylene glycol)-5000]-peptide DSPE-PEG5k-peptide). The RES-NPs are around 100 nanometers in diameter. In addition, the RES is held in place by vitamin E acetate. The DSPE-PEG5k-peptide helps target the adipose tissue and attaches to a receptor on the ASC. Also shown is a chemical structure of RES.

FIG. 1C provides a scheme of a method for delivering one or more active agents to adipose stromal cells through the use of delivery agents.

FIG. 1D shows that the local and targeted delivery of ASC-targeted RES-NPs to mouse iBAT (interscapular brown adipose tissue) and iWAT (inguinal white adipose tissue) increases the amount of BAT and beige cells and their thermogenic activities, and improves metabolic activities. This occurs through a process where the ASC-targeted RES-NPs target both brown adipose tissue and white adipose tissue, which attaches itself to ASCs via a receptor (FIG. 1D-A). Once in the cell (FIG. 1D-B), RES is released and used to induce brown and brown-like adipocyte formation (FIG. 1D-C).

FIG. 1E shows that the same process as illustrated in FIG. 1D can be conducted in human subjects.

FIG. 1F illustrates various working mechanisms of ASC-targeted RES-NPs.

FIG. 1G provides images of additional delivery agents for delivering RES and other active agents into adipose stromal cells.

FIG. 1H illustrates the different types of active agents that can be carried by the particles of the present disclosure.

FIG. 1I illustrates the differentiation potential of ASCs, and how ASCs can be used as targets for various diseases or disorders.

FIG. 2 shows the characteristics of RES encapsulated lipid nanocarriers (Rnano) and R encapsulated liposomes (R-lipo). FIG. 2A shows the visual observation of Rnano, R-lipo, and native RES (R) containing 1 mg of R suspended in 1 mL of 1×PBS, transmission electron microscope (TEM) images of Rnano and R-lipo, and predicted structures of Rnano and R-lipo. R-lipo can have multiple phospholipid bilayers. FIG. 2B shows changes of particle size, zeta potential, and the polydispersity index of Rnano and R-lipo at different temperatures.

FIG. 3 shows the chemical stability of native R, Rnano, and R-lipo under light (FIG. 3A) or dark (FIG. 3B) at different temperatures.

FIG. 4 shows various physicochemical characterizations. Shown are Raman spectra, X-ray diffraction patterns, differential scanning calorimetry (DSC) thermograms of lyophilized Rnano or R-lipo; lyophilized void nanocarriers (V-nano) or void liposomes (V-lipo); and native R.

FIG. 5 shows in vitro release profiles, including hourly (FIG. 5A) and accumulative (FIG. 5B) R release for native R, Rnano, and R-lipo.

FIG. 6 shows the cytotoxicity and R content in 3T3-L1 cells. Cytotoxicity was measured by colorimetric 3-(4,5-dimethylthiazolyl-2)-2,5-diphenyltetrazolium bromide (MTT) assays after treating 3T3-L1 cells with native R, R-lipo, and Rnano (5, 10, 20 μM) or their respective controls for 24 hr and 72 hr (FIG. 6A); and R content in 3T3-L1 cells after treating them with 10 and 20 μM of native R, Rnano and R-lipo at 37° C. for 4 hr (FIG. 6B). Data=mean±standard error of the mean (SEM) (n=3). *, p<0.05. E, Ethanol containing vehicle control for native R. R, rosiglitazone, a positive control.

FIG. 7 shows activation of the peroxisome-proliferator-activated receptor (PPAR) responsive reporter by various forms of R. 3T3-L1 cells were seeded and transiently transfected with a peroxisome proliferator response element (PPRE)-driven luciferase reporter (PPRE-Luc) and transfection control plasmid P-gal for 24 hr. The cells were then treated with native R, R-lipo, and Rnano (5, 10, 20 μM) and their controls for 15 hr. Relative luciferase activities were normalized by β-gal activities. Data=mean±SEM (n=3). E, Ethanol containing vehicle control for native R. R, rosiglitazone, a positive control. Different letters on top of the bars indicate significant differences among various forms of R. *, p<0.05; **, p<0.01 compared to their controls.

FIG. 8 shows browning activities. 3T3-L1 cells were induced to undergo white adipocyte differentiation in the presence of native R, R-lipo, and Rnano (5, 10, 20 μM) and their controls for 7 days. The cells were then stimulated with isoproterenol (ISO) for 6 hr. Relative mRNA expression of Uncoupling protein 1 (UCP-1), PPARγ, PPARγ co-activator 1α (PGC-1α), and PR-domain-containing 16 (PRDM16) were analyzed by semi-quantitative Real Time-PCR. Data=mean±SEM (n=3). E, Ethanol containing vehicle control for native R. R, rosiglitazone, a positive control. Letters of a-c or a′-c′ on top of the bars define differences among various forms of R under either basal or ISO-stimulated conditions, respectively. Different letters indicate significant differences among various R. *, p<0.05; **, p<0.01; ***, p<0.001 compared to their controls.

FIG. 9 shows the gene expression of white and beige adipocyte markers. 3T3-L1 cells were induced to undergo white adipocyte differentiation in the presence of native R, R-lipo, and Rnano (5, 10, 20 μM) and their controls for 7 days. The cells were then stimulated with ISO for 6 hr. Relative mRNA expression of white marker insulin-like growth factor-binding protein 3 (IGFBP3) (FIG. 9A) and beige marker CD137 and transmembrane protein 26 (Tmem26) (FIG. 9B) were analyzed by semi-quantitative reverse transcription polymerase chain reaction (RT-PCR). Data=mean±SEM (n=3). E, Ethanol containing vehicle control for native R. R, rosiglitazone, a positive control. Letters of a-c or a′-c′ on top of the bars define differences among various forms of R under both basal and ISO-stimulated conditions, respectively. Different letters indicate significant differences among various R. *, p<0.05; **, p<0.01 compared to the controls.

FIG. 10 shows RES-NP signals in mice (FIG. 10A) and isolated adipose tissue and livers (FIG. 10B) detected using an IVIS® Lumina XR imaging system. ASC target specificity was detected using flow cytometry (FIGS. 10C-1 and 10C-2). In particular, FIG. 10C-2 shows data from in vitro binding test (delta-DCN cells)-flow cytometer.

FIG. 11 shows visual observation of free RES and RES-NPs containing 1 mg of RES suspended in 1 mL of 1×PBS (FIG. 11A); transmission electron microscope (TEM) images of RES-NPs (FIG. 11B); body weight (FIG. 11C), percentage of body fat (FIG. 11D), percentage of body lean mass (FIG. 11E) and iWAT weight (FIG. 11F) of C57BL/6J mice after receiving intravenous injection of the treatments for 5 weeks. Also shown are H&E staining of cross-sections of iWAT (FIG. 11G); UCP-1 gene expression in iWAT (FIG. 11H); and UCP-1 protein expression in iWAT (FIG. 11) isolated from these mice. The study was conducted by treating obese C57BL/6J mice with saline control (treatment 1), 15 mg/kg body weight daily dose free RES (treatment 2), 15 mg/kg non-targeted RES-NPs (treatment 3), and 15 mg/kg ASC-targeted RES-NPs (treatment 4) via tail vein injection twice per week for 5 weeks (5 mice per treatment group).

FIG. 12 provides a structure of DSPE-PEG₅₀₀₀-peptide.

FIG. 13 shows matrix-assisted laser desorption/ionization time of flight mass spectrometry (MALDI-TOF MS) chromatograms of DSPE-PEG₅₀₀₀-maleimide (MW≈5580), peptide (MW: 1376) and DSPE-PEG₅₀₀₀-peptide (MW≈6956).

FIG. 14 shows changes in particle size, polydispersity index (PI) and zeta potential of Rnano (FIG. 14A) and ligand-coated Rnano (L-Rnano) (FIG. 14B) at different temperatures.

FIG. 15 shows in vitro release profiles for free R, Rnano and L-Rnano in release media. The profiles shown in FIGS. 15A and 15B represent different formulas of Rnano and L-Rnano.

FIG. 16 shows representative fluorescence images of ΔDCN cells after treating them with Rhoda-labeled L-Vnano, Vnano, L-Rnano and Rnano for 2 hours at either 37° C. or 4° C. 3T3-L1 cells have been used as a control. Cell nuclei were stained with DAPI and overlaid with fluorescent images of Rhoda. Images represent three independent experiments.

FIG. 17 shows a gating strategy for ΔDCN cells treated with 1, 1″-dioctadecyl-3, 3, 3″, 3″-tetramethylindodicarbocyanine, 4-chlorobenzenesulfonate (DiD)-L-Rnano, DiD-Rnano and saline, which are diagramed from left to right. In the first step, ΔDCN cells were identified by size in a dot plot of forward scatter (FSC) versus side scatter (SSC). In the second step, the events containing DiD were gated. The percentage of population stained by DiD was gated from the unstained population.

FIG. 18 shows R content in ΔDCN cells after treating them with free R, Rnano and L-Rnano at 37° C. and 4° C. for 4 hours. *, p<0.05; **, p<0.01.

FIG. 19 shows DiD fluorescence images of C57BL/6J mice and isolated fat pads after treating them with DiD-labeled Vnano and ligand coated Vnano (L-Vnano) (FIG. 19A), and DiD-labeled Rnano and L-Rnano (FIG. 19B) by an IVIS® Lumina XR imaging system.

FIG. 20 shows gating and analyzing strategies for WAT stromal vascular fractions (SVF) and mature adipocytes isolated from the C57BL/6J mice's I-WAT (FIG. 20A) and gonadal WAT (G-WAT) (FIG. 20B) that were treated with DiD-labeled L-Rnano and Rnano. In the first step, the SVF population was identified by size in a dot plot of FSC versus SSC. In the second step, the CD45⁻CD31⁻ events were gated from the histogram plot. In the third step, the CD45⁻CD31⁻CD34⁺ events were gated from the CD45⁻CD31⁻ population. In the fourth step, the CD45⁻CD31⁻CD34⁺CD29⁺ event, which was the population of ASC, were gated from the CD45⁻CD31⁻CD34⁺ population.

FIG. 21 shows body weight, body composition and food intake changes of C57BL/6J mice from each treatment during 5 weeks. Shown are body weight changes (FIG. 21A); weekly food intake changes (FIG. 21B); percent body fat and fat mass weight changes at week 5 (FIG. 21C) and percent body lean and lean mass weight at week 5 (FIG. 21D) in C57BL/6 mice of each treatment. Values are mean±SEM, n=9 to 10 per treatment, *, p<0.05; **, p<0.01; ***, p<0.001. FIG. 21E shows representative abdominal views of the fat pads of mice after 5 weeks of treatment.

FIG. 22 shows changes in mice core body temperature during 6 hours of cold exposure at 4° C. Values are mean±SEM, n=5 per treatment. FIG. 22A shows a chart that illustrates the core body temperature changes. FIG. 22B shows the core body temperature changes as areas under the curve (AUC).

FIG. 23 shows tissue weights of G-WAT, I-WAT, retroperitoneal WAT (RP-WAT), and BAT isolated from mice after each treatment. Values are mean±SEM, n=9 to 10 per treatment *, p<0.05; **, p<0.01; ***, p<0.001.

FIG. 24 shows fasting plasma insulin concentrations (FIG. 24A); glucose concentrations (FIG. 24B); and homeostatic model assessment of insulin resistance (HOMA-IR) values (FIG. 24C), which were measured and calculated after sacrifice. Values are means±SEM, n=9 to 10 per treatment. Also shown are glucose tolerance tests (GTT) and area under the curve for GTT (FIG. 24D), and insulin tolerance test (ITT) and area under the curve for ITT (FIG. 24E) performed on week 4 and week 5 of treatment, respectively. Values are mean±SEM, n=5 per treatment.

FIG. 25 shows RT-PCR analysis of thermogenic gene expression of UCP-1 in I-WAT (FIG. 25A); PGC-1α, PRDM16, PPAR-γ, CD137 and TMEM26 in I-WAT (FIG. 25B); adipokine gene expression of leptin and adiponectin in I-WAT (FIG. 25C); inflammation markers of interleukin 6 (IL-6), monocyte chemoattractant protein-1 (MCP-1) and tumor necrosis factor alpha (TNF-α) (FIG. 25D) in I-WAT; and UCP-1 gene expression in G-WAT (FIG. 25E) and BAT (FIG. 25F). Values are mean±SEM, n=7 to 9 per treatment. The plasma concentrations of TNF-α, MCP-1, IL-6, and IFN-γ are shown in FIG. 25G. The measured F4/80 mRNA levels in I-WAT are shown in FIG. 25H.

FIG. 26 shows blood lipid profile in mice after different treatments, including levels of triglyceride (TG) (FIG. 26A); total cholesterol (TC) (FIG. 26B); high-density lipoprotein cholesterol (HDL-C) (FIG. 26C); low-density lipoprotein cholesterol (LDL-C) (FIG. 26D); and very low-density lipoprotein cholesterol (VLDL-C) (FIG. 26E). Values are means±SEM, n=6 per treatment.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Obesity and its related metabolic disorders have become a major global public health problem. Obesity is characterized by an increase in the fat mass of a person. Currently, one in three adults is obese, and two in three adults in the United States are either obese or overweight.

There are a number of diseases that are positively associated with obesity. Such diseases include type 2 diabetes, cardiovascular diseases, hypertension, as well as some types of cancers. While there are a number of treatment plans for obesity, many are either invasive or require strict personal discipline.

The most common obesity treatment is a healthy lifestyle change, which includes healthy eating and exercise habits. The healthy lifestyle change method requires an extreme amount of personal discipline. Moreover, many individuals who are able to achieve weight loss usually gain the weight back.

Surgical methods for controlling obesity include gastric bypass and gastric banding. These methods have been shown to be effective but are extremely invasive, costly, and require a certain level of lifestyle change. As such, a need exists for a low-cost, non-invasive, and safe obesity treatments.

Pharmacotherapy utilizes orally administered drugs. Most Food and Drug Administration (FDA) approved drugs target energy intake either by suppressing appetite (e.g., Phentermine) or by interfering with nutrient absorption (e.g., Orlistat). Orally administered drugs have the highest compliance but are beset with major problems such as a high level of hepatic metabolism (the first-pass effect) and a low level of target specificity, leading to a high level of side effects and toxicity. Obesity relapse may also occur when drugs are stopped.

A person's fat mass is made up of adipocytes that can be categorized into two groups known as white adipose tissue (WAT) and brown adipose tissue (BAT), including beige adipose tissue. WAT is used by the body for energy storage (fat reserves), and BAT is used for thermogenesis (the production of body heat) and energy expenditure. Adipose stromal stem cells (ASC) are able to differentiate into either WAT or BAT depending on certain factors and needs.

Morphologically and functionally distinct from WAT, and specialized in storing energy as triglycerides, BAT utilizes its high amount of mitochondria and uncoupling protein 1 (UCP-1) to dissipate the proton electrochemical gradient generated from oxidative phosphorylation in the form of heat. Although it has been believed for many years that BAT is a therapeutic target for treating obesity, recent studies have shown that adults do not possess eligible or active BAT, and BAT decreases or disappears with aging.

Beige adipose tissue has the similar brownish characteristics and thermogenic functions as BAT. Beige adipocytes are inducible in WAT by certain types of stimuli, such as cold, pharmacological and nutritional agents and other stimuli, via the de novo differentiation of ASCs, and through the promotion of mitochondrial UCP-1 expression, causing WAT browning and contributing to extra energy consumption and burning.

As mesenchymal progenitors found in the stromal vascular fractions (SVFs) of WAT, ASCs have multipotent differentiation capacities. Furthermore, ASC's brown adipogenic potential through activating related regulatory transcription factors and pathways have been investigated and evaluated by many studies. These ASCs can also be differentiated into brown-like/beige adipocytes after receiving appropriate cues in the adipose tissue. The induced brown-like/beige adipocytes have the same thermogenesis and metabolic sink functions as classical brown adipocytes. Thus, enhancement of beige adipocytes formation in human WAT might be a feasible and efficient approach for combating obesity and its related metabolic diseases.

Various active agents may be utilized to treat or prevent obesity. For instance, resveratrol (3,5,4′-trihydroxy-trans-stilbene) is a type of naturally occurring phenol that is produced by several plants in response to pathogen attack. The most commonly known sources of resveratrol are the skins of grapes, blueberries, raspberries, and mulberries. Interestingly, resveratrol has been shown to lower the severity of obesity.

Resveratrol has demonstrated the ability to increase the amount of BAT tissue that is produced from ASCs, as well as potentially convert pure WAT into brown-like adipose tissue, which has characteristics of both WAT and BAT. The increase in BAT and brown-like adipose tissue results in more energy expenditure and less storage of fat throughout a person's body.

Unfortunately, many active agents that are utilized to treat or prevent obesity are unable to reach fat cells in an effective manner. For instance, resveratrol has low aqueous solubility, poor target (e.g., ASC) specificity, and high hepatic metabolization, which cause resveratrol alone to be relatively ineffective.

As such, a need exists for new compositions and methods of delivering active agents into adipose stromal cells. Embodiments of the present disclosure address the aforementioned need.

In some embodiments, the present disclosure pertains delivery agents for delivering one or more active agents to adipose stromal cells. In some embodiments, the delivery agents include at least the following components: (1) a particle; (2) one or more active agents carried by the particle; and (3) a targeting agent associated with the particle. In some embodiments, the targeting agent directs the delivery agent to the adipose stromal cells.

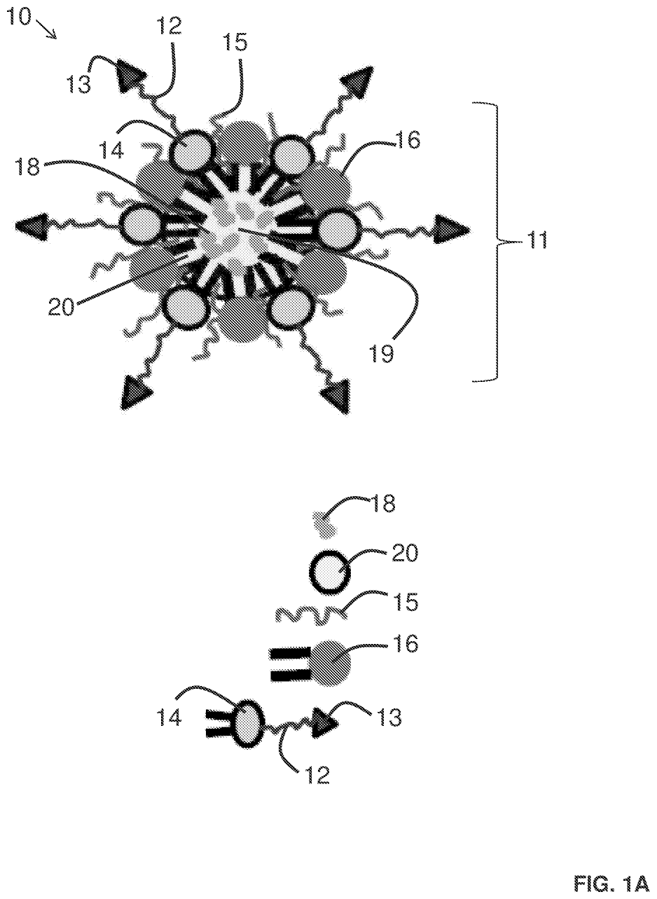

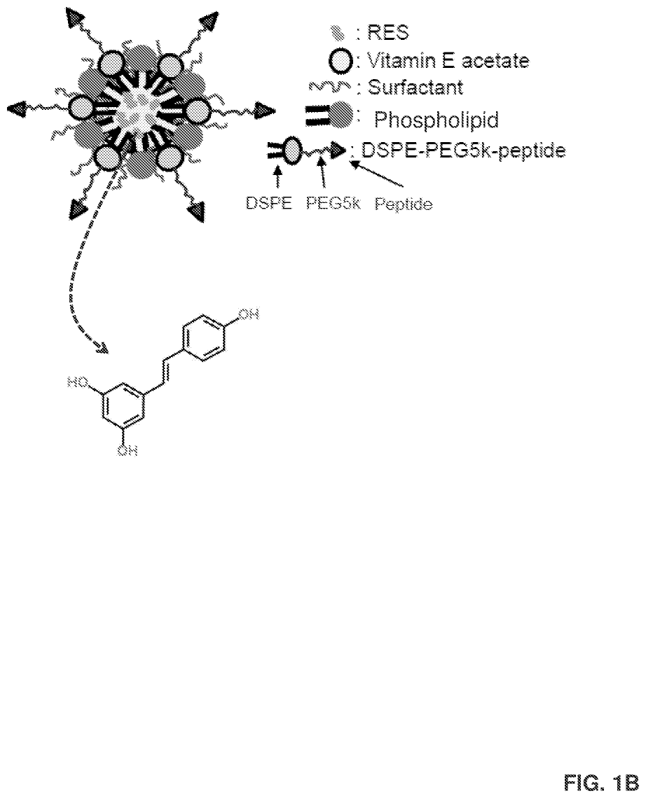

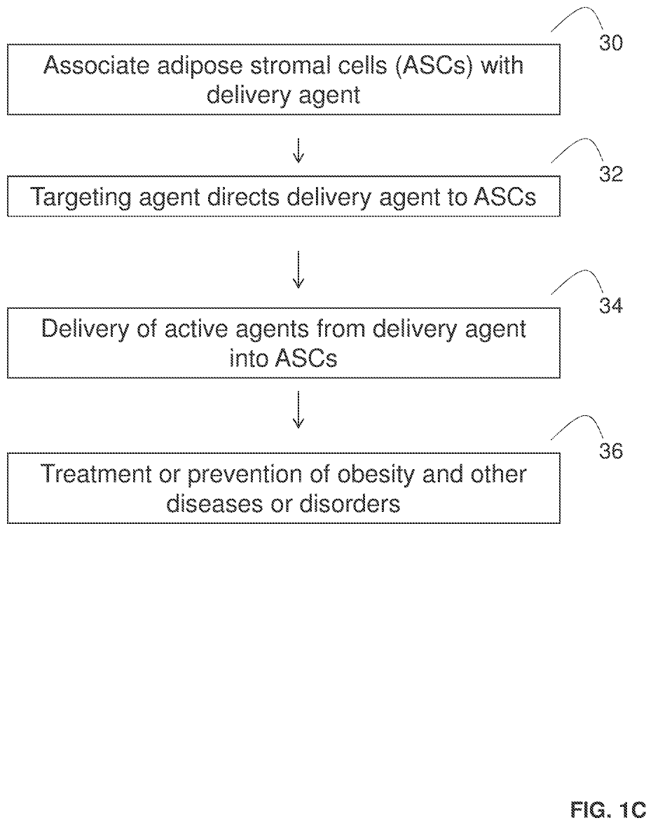

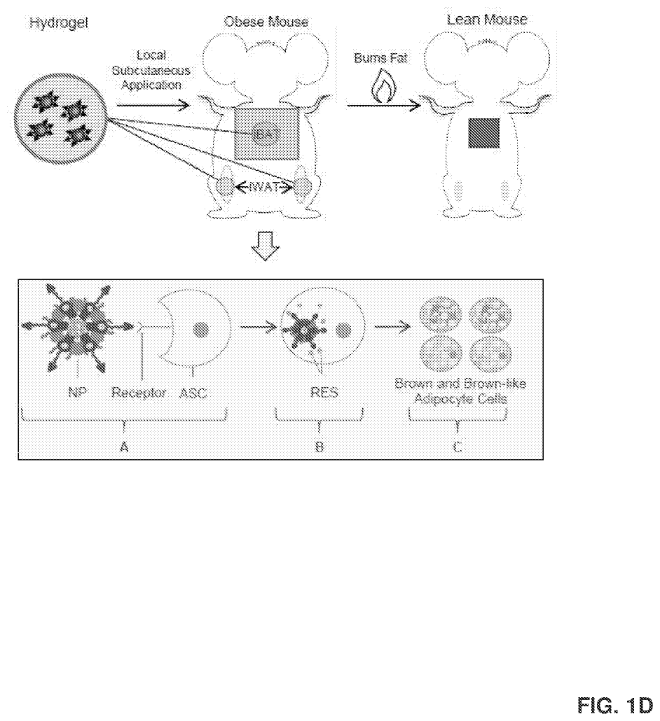

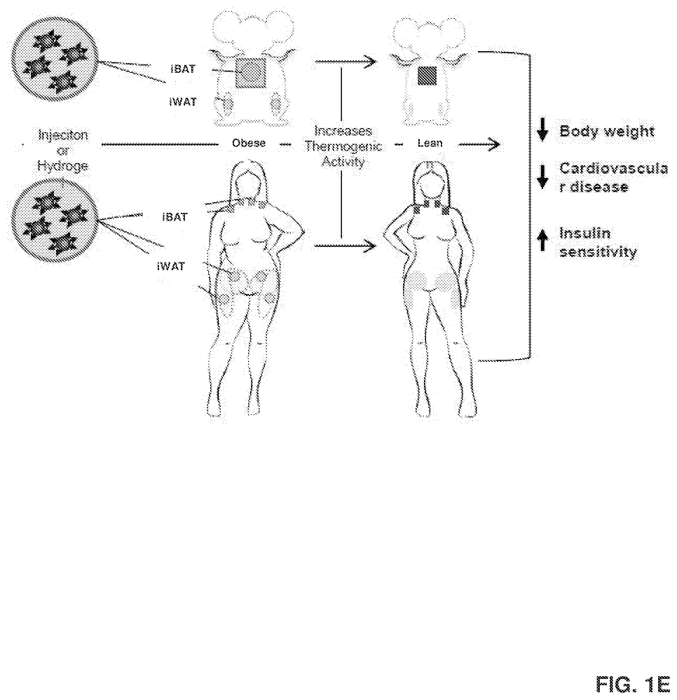

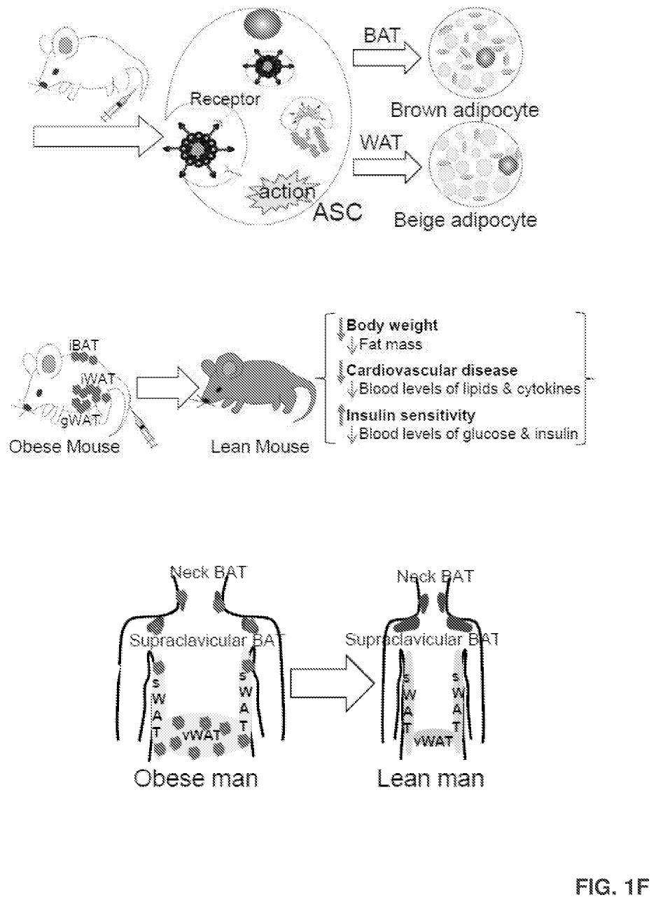

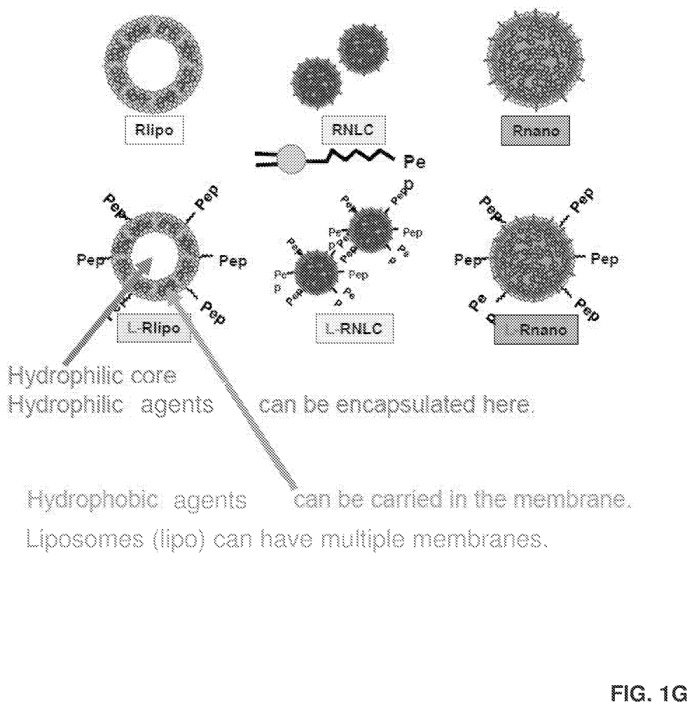

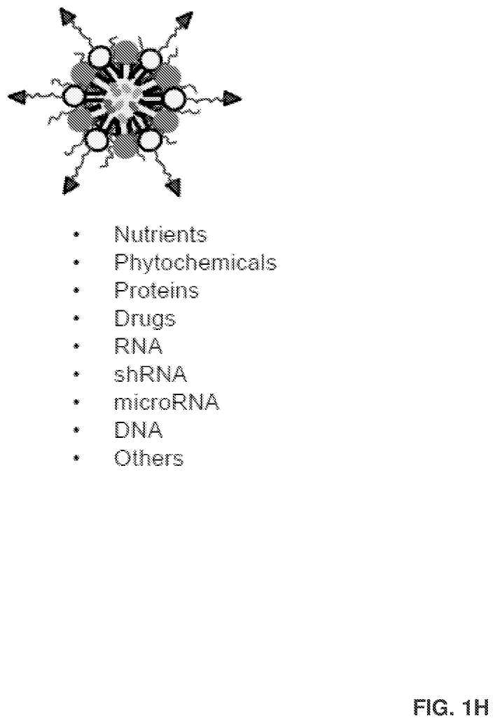

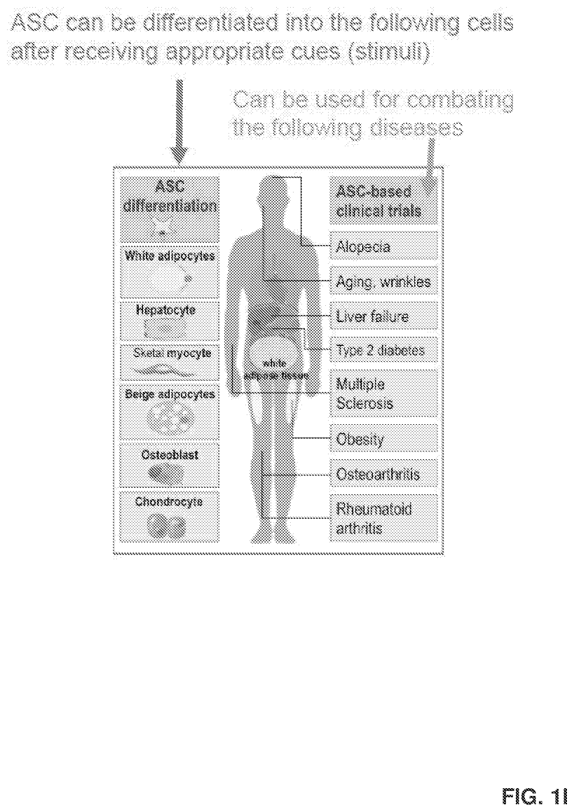

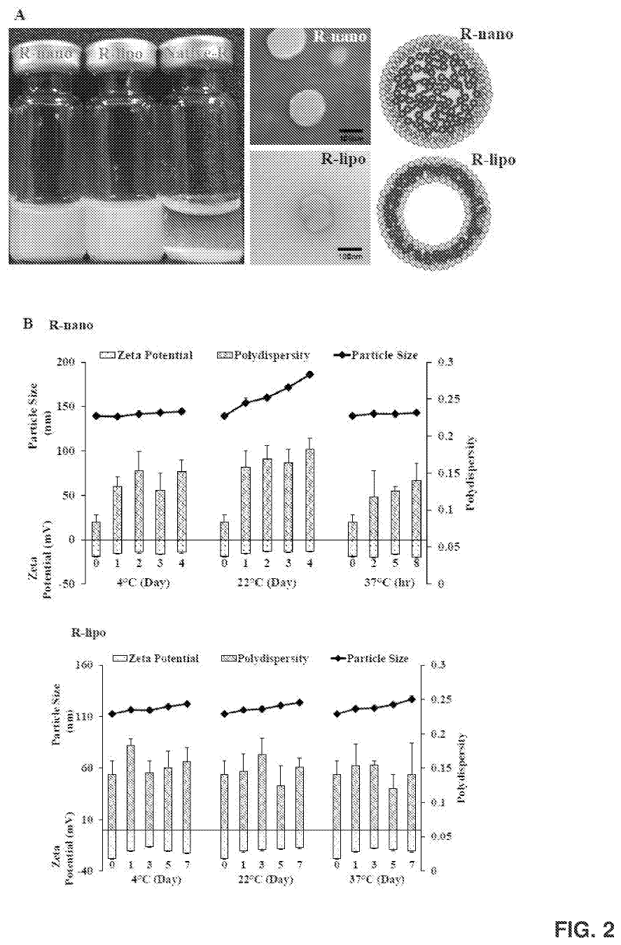

A more specific embodiment of a delivery agent is illustrated in FIG. 1A as delivery agent 10. In this embodiment, delivery agent 10 is in the form of particle 11, which includes: phospholipids 14 and 16 that form the particle; a core region 19, active agents 18 encapsulated within the core region of the particle; active agent stabilizer 20 for stabilizing the active agent; surfactants 15 on the surface of the particle for lowering the surface tension of the particle; and targeting agent 13 associated with the surface of the particle for directing the delivery agent to desired cells, such as adipose stromal cells. In this example, targeting agent 13 is associated with particle 11 through a linker 12 that couples the targeting agent to phospholipid 14.

Another specific embodiment of a delivery agent is illustrated in FIG. 1B as a delivery agent for delivering resveratrol into adipose stromal cells. In this embodiment, the delivery agent is in the form of a phospholipid-based particle (e.g., phosphatidylcholine-based particle) that encapsulates resveratrol within the core of the particle. The particle also includes vitamin E acetate within the particle as an active agent stabilizer for stabilizing the resveratrol, and a surfactant (e.g., Kolliphor® HS15) on a surface of the particle for lowering the surface tension of the particle. In addition, the particle includes a peptide-based targeting agent on a surface of the particle for directing the delivery agent to adipose stromal cells. In this example, the peptide-based targeting agent is associated with a surface of the particle through a polyethylene glycol-based linker that couples the peptide-based targeting agent to phospholipids on the surface of the particle.

In some embodiments, the present disclosure pertains to methods of utilizing the delivery agents of the present disclosure to deliver one or more active agents to adipose stromal cells. In some embodiments illustrated in FIG. 1C, the methods of the present disclosure include a step of associating the adipose stromal cells with the delivery agent (step 30) such that the targeting agent directs the delivery agent to the adipose stromal cells (step 32) to result in the delivery of the one or more active agents into the adipose stromal cells (step 34). In some embodiments, the delivery of one or more active agents into the adipose stromal cells can have various therapeutic applications, such as treatment or prevention of obesity and other disorder or diseases (step 36).

As set forth in more detail herein, the methods and delivery agents of the present disclosure can have numerous embodiments. For instance, the delivery agents of the present disclosure can include various types of particles and targeting agents. Furthermore, various active agents may be associated with the particles in various manners. Moreover, the delivery agents and methods of the present disclosure may target various types of adipose stromal cells through various mechanisms and for various purposes. In addition, the delivery agents of the present disclosure may be in various forms.

### Particles

In the present disclosure, particles are not limited to any particular shapes, compositions or sizes. In particular, the delivery agents of the present disclosure can include various types of particles with various compositions, properties, and sizes that are suitable for delivering one or more active agents to desired cells. In addition, in various embodiments, the particles of the present disclosure may include various active agent stabilizers and surfactants.

### Particle Compositions

The particles of the present disclosure can include various compositions. For instance, in some embodiments, the particles of the present disclosure include lipid-based particles, carbon-based particles, metal-based particles, and combinations thereof.

In some embodiments, the particles of the present disclosure include lipid-based particles. In some embodiment, the lipid-based particles include phospholipids (e.g., phospholipids 14 and 16 shown in FIG. 1A). In some embodiments, the phospholipids include, without limitation, lecithin, phosphatidylcholine, phosphatidic acid, phosphatidylethanolamine, phosphatidylserine, phosphoinositides, phosphatidylinositol, phosphatidylinositol phosphate, phosphatidylinositol bisphosphate, phosphatidylinositol trisphosphate, ceramide phosphorylcholine, ceramide phosphorylethanolamine, ceramide phosphoryllipid, derivatives of phospholipids, and combinations thereof. In some embodiments, the phospholipids of the present disclosure include phosphatidylcholine. In some embodiments, the phospholipids of the present disclosure include phospholipid derivatives.

The lipid-based particles of the present disclosure may be in various forms. For instance, in some embodiments, the lipid-based particles of the present disclosure may be in the form of liposomes.

In some embodiments, the lipid-based particles of the present disclosure include a lipid membrane. In some embodiments, the lipid membrane is a lipid bilayer membrane. In some embodiments, the lipid membrane is a lipid monolayer membrane. In some embodiments, the particles have multiple membranes.

In some embodiments, the particles of the present disclosure contain triglycerides. In some embodiments, the particles of the present disclosure (e.g., lipid-based particles) lack triglycerides. In some embodiments, triglycerides from the particles of the present disclosure (e.g., lipid-based particles) are replaced by an active agent stabilizer (e.g. vitamin E) or other hydrophobic agents, thereby eliminating exogenous triglyceride and increasing the anti-oxidative capacity of nanocarriers.

### Particle Properties

The particles of the present disclosure may have various properties. For instance, in some embodiments, the particles of the present disclosure include a surface with a negative charge. In some embodiments, the particles of the present disclosure include a surface with a positive charge. In some embodiments, the particles of the present disclosure include a surface with a neutral charge.

In some embodiments, the particles of the present disclosure include a hydrophobic core. In some embodiments, the particles of the present disclosure include a hydrophilic core. In some embodiments, the particles of the present disclosure include a neutral core. In some embodiments, the particles of the present disclosure include an amphiphilic core.

In some embodiments, the particle cores of the present disclosure have the same properties as the active agents of the present disclosure. For instance, in some embodiments, both the particle core and the active agents are hydrophobic. In some embodiments, both the particle core and the active agents are hydrophilic.

### Particle Sizes and Shapes

The particles of the present disclosure may have also various sizes. For instance, in some embodiments, the particles of the present disclosure are in the form of nanoparticles. In some embodiments, the nanoparticles have diameters ranging from about 1 nm to about 5000 nm. In some embodiments, the nanoparticles have a diameter of about 150 nm to about 5000 nm. In some embodiments, the nanoparticles have diameters of about 50 nm to about 500 nm. In some embodiments, the nanoparticles have diameters of about 100 nm to about 150 nm. In some embodiments, the nanoparticles have diameters of about 1 nm to about 100 nm. In some embodiments, the nanoparticles have diameters of about 20 nm to about 200 nm. In some embodiments, the nanoparticles have a diameter of about 100 nm. As used herein, a diameter refers to a length from one end of a particle to another end of the particle on any dimensions.

The particles of the present disclosure may have also various shapes. For instance, in some embodiments, the particles of the present disclosure have a spherical shape. In some embodiments, the particles of the present disclosure have a cylindrical shape. In some embodiments, the particles of the present disclosure have a circular shape. In some embodiments, the particles of the present disclosure have an elliptical shape. Additional particle shapes suitable for delivering one or more active agents to desired cells can also be envisioned.

In some embodiments, a single type of particle that contains one or more active agents is utilized in a delivery agent. In some embodiments, two or more different types of particles that each contain one or more of the same or different active agents are utilized in a delivery agent.

### Active Agent Stabilizers and Excipients

In some embodiments, the particles of the present disclosure also include one or more active agent stabilizers. Active agent stabilizers generally refer to compounds that are capable of reducing or preventing the degradation of the active agents of the present disclosure.

In some embodiments, the active agent stabilizers of the present disclosure include, without limitation, anti-oxidants, sequestrants, ultraviolet stabilizers, and combinations thereof.

In some embodiments, the active agent stabilizers of the present disclosure include anti-oxidants. In some embodiments, the anti-oxidants include, without limitation, vitamin E, vitamin C, triglyceride, lipids, cellulose, fibers, uric acid, glutathione, and combinations thereof. In some embodiments, the active agent stabilizers of the present disclosure include vitamin E.

In some embodiments, the active agent stabilizers of the present disclosure include sequestrants. In some embodiments, the sequestrants include, without limitation, calcium chloride, calcium acetate, calcium disodium ethylene diamine tetra-acetate, glucono delta-lactone, sodium gluconate, potassium gluconate, sodium tripolyphosphate, sodium hexametaphosphate, ethylenediaminetetraacetic acid (EDTA), and combinations thereof.

In some embodiments, the active agent stabilizers of the present disclosure include ultraviolet stabilizers. In some embodiments, the ultraviolet stabilizers include benzophenones.

The use of additional active agent stabilizers and excipients can also be envisioned. For instance, in some embodiments, the active agent stabilizers and excipients can include triglycerides and/or other agents that have different melting temperatures. In some embodiments, the active agent stabilizers and excipients, can include, but are not limited to, monosaccharides, disaccharides, polysaccharides, fibers, lipids, vitamins, minerals, phytochemicals, proteins and terpenoids.

The active agent stabilizers and excipients of the present disclosure may be associated with the active agents of the present disclosure in various manners. For instance, in some embodiments, the active agent stabilizers and excipients of the present disclosure may be co-encapsulated with the active agents of the present disclosure within the particles of the present disclosure. In some embodiments, the active agent stabilizers and excipients of the present disclosure may be non-covalently associated with the active agents of the present disclosure. In some embodiments, the active agents of the present disclosure may be held in place by active agent stabilizers of the present disclosure within a particle core.

### Surfactants

In some embodiments, the particles of the present disclosure also include one or more surfactants. Surfactants generally refer to compounds that are capable of lowering the surface tension of the particles of the present disclosure. In some embodiments, the surfactants include, without limitation, anionic surfactants, cationic surfactants, zwitterionic surfactants, non-ionic surfactants, and combinations thereof.

In some embodiments, the surfactants of the present disclosure include non-ionic surfactants. In some embodiments, the non-ionic surfactants include, without limitation, ethoxylates, fatty acid esters of polyhydroxy compounds, amine oxides, sulfoxides, phosphine oxides, and combinations thereof.

In some embodiments, the surfactants of the present disclosure include, without limitation, octaethylene glycol monododecyl ether, pentaethylene glycol monododecyl ether, nonoxynols, polyethylene glycol, Triton X-100, polyethoxylated tallow amine, cocamide monoethanolamine, cocamide diethanolamine, poloxamers, glycerol monostearate, glycerol monolaurate, sorbitan monolaurate, sorbitan monostearate, sorbitan tristearate, Tween 20, Tween 40, Tween 60, Tween 80, decyl glucoside, lauryl glucoside, octyl glucoside, lauryldimethylamine oxide, dimethyl sulfoxide, phosphine oxide, polyoxyl hydroxystearates, and combinations thereof.

In some embodiments, the surfactant is polyoxyl 15 hydroxystearate (i.e., Kolliphor® HS15). The use of additional surfactants can also be envisioned.

The surfactants of the present disclosure may be associated with particles in various manners. For instance, in some embodiments, the surfactants of the present disclosure are on a surface of a particle. In some embodiments, the surfactants of the present disclosure are embedded with a particle layer on a surface of a particle. In more specific embodiments, the surfactants of the present disclosure are embedded with a phospholipid layer on a surface of a lipid-based particle.

### Active Agents

Various types of active agents may be carried by the particles of the present disclosure. In some embodiments, the active agents include active agents that can be utilized to treat or prevent obesity. In some embodiments, the active agents include, without limitation, small molecules, peptides, polypeptides, proteins, hydrophobic active agents, hydrophilic active agents, drugs, nucleotides, RNA, shRNA, siRNA, miRNA, DNA, nutrients, phytochemicals, and combinations thereof. In some embodiments, the active agents include hydrophobic active agents. In some embodiments, the active agents include hydrophilic active agents. In some embodiments, the active agents include amphiphilic active agents.

In some embodiments, the active agents include bioactive compounds. In some embodiments, the active agents include resveratrol. In some embodiments, the active agents include alpha-tocopherol acetate. In some embodiments, the active agents include retinoic acids. In some embodiments, the active agents include peroxisome-proliferator-activated receptor (PPAR) agonists. In some embodiments, the PPAR agonists include, without limitation, thiazolidinedione, picoglitazone, rosiglitazone, lobeglitazone, and combinations thereof. In some embodiments, the active agents include pharmaceutical agents (i.e., PPARgamma agonists, PPARalpha agonists, metformin, beta-adrenergic receptor agonists, and 5′ AMP-activated protein kinase (AMPK) activators), dietary factors (i.e., resveratrol, berberin, capsaicin and capsaicin-analogs, n-3 fatty acids and their derivatives) and other endogenous bioactive molecules (i.e., irisin, thyroid hormone, T3, natriuretic peptides (NP), fibroblast growth factor 21 (FGF21), bone morphogenetic protein 7 (BMP7), bone morphogenetic protein 8b (BMP8b), orexin (OX), vascular endothelial growth factor (VEGF) and prostaglandins (PG) T3, FGF21, BMP7), meteorin-like (METRNL), interleukin 6 (IL-6), lactate, norepinephrine (NE) and O-aminoisobutyric acid (BAIBA))

In some embodiments, the active agents include one or more miRNAs (i.e., microRNAs or miR). In some embodiments, the miRNAs include, without limitation, miR-32, miR-155, and combinations thereof.

In some embodiments, the active agents include one or more of the bioactive compounds disclosed in U.S. Pat. Nos. 8,00,8436; 8,951,980; 9,346,835; 9,469,659; 9,714,259; and 9,433,659 (e.g., Adenovirus 36 E4 ORF1 proteins, nucleic acids, and small molecule analogues). In some embodiments, the active agents include the bioactive compounds disclosed in U.S. patent application Ser. No. 15/305,479 (e.g., Adenovirus 36 E4 ORF1 protein small molecule analogues). The use of additional active agents can also be envisioned.

Active agents may be carried by the particles of the present disclosure in various manners. For instance, in some embodiments, the active agents are encapsulated within the particle. In some embodiments, the active agents are within the core of the particles (e.g., the hydrophobic or hydrophilic core of particles). In some embodiments, the active agents are within a layer or membrane of the particles. In some embodiments, the active agents are within the lipid membrane of the particles. In some embodiments, the active agents are dispersed within the particle in the form of an amorphous phase.

The particles of the present disclosure may include various concentrations of active agents. For instance, in some embodiments, the particles of the present disclosure include an active agent concentration of more than 1 nM. In some embodiments, the particles of the present disclosure include an active agent concentration of more than 500 nM. In some embodiments, the particles of the present disclosure include an active agent concentration of more than 1 μM. In some embodiments, the particles of the present disclosure include an active agent concentration of more than 2 μM. In some embodiments, the particles of the present disclosure include an active agent concentration of about 5 μM. In some embodiments, the particles of the present disclosure include an active agent concentration of about 10 μM. In some embodiments, the particles of the present disclosure include an active agent concentration of about 15 μM. In some embodiments, the particles of the present disclosure include an active agent concentration of about 20 μM. In some embodiments, the particles of the present disclosure include an active agent concentration of about 25 μM. In some embodiments, the particles of the present disclosure include an active agent concentration of about 5-50 μM.

In some embodiments, the particles of the present disclosure include a single active agent. In some embodiments, the particles of the present disclosure include a plurality of active agents. In some embodiments, the plurality of active agents act in a synergistic manner to treat or prevent a disease or disorder.

### Targeting Agents

The delivery agents of the present disclosure may include various targeting agents. Targeting agents generally refer to compounds or compositions that are able to direct the delivery agents of the present disclosure to desired cells, such as adipose stromal cells. In some embodiments, the targeting agents of the present disclosure include, without limitation, amino acids, peptides, proteins, aptamers, antibodies, small targeted particles, carbohydrates, polysaccharides, lipids, and combinations thereof.

In some embodiments, the targeting agent is a peptide. In some embodiments, the peptide is a linear peptide or a cyclic peptide. In some embodiments, the targeting agent is a peptide (e.g., a linear or cyclic peptide) that directs the delivery agents of the present disclosure to adipose stromal cells. In some embodiments, the particles of the present disclosure include a single type of peptide as a targeting agent. In some embodiments, the particles of the present disclosure include a plurality of different types of peptides as a targeting agent.

In some embodiments, the peptide includes the following sequence: CSWKYWFGEC (WAT 7) (SEQ ID NO: 1). In some embodiments, the peptide (e.g., a linear or cyclic peptide) includes the following sequence: GSWKYWFGEGGC (SEQ ID NO: 2).

In some embodiments, the targeting agent is a peptide with naturally occurring amino acids. In some embodiments, the targeting agent is a peptide with non-naturally occurring amino acids, such as non-canonical amino acids.

In some embodiments, additional amino acids can be added to a peptide that has been attached to a surface of a particle. In some embodiments, amino acids on a peptide can be replaced with amino acids with similar characteristics.

The targeting agents of the present disclosure may be associated with the particles of the present disclosure in various manners. For instance, in some embodiments, the targeting agents of the present disclosure may be on a surface of a particle. In some embodiments, the targeting agents of the present disclosure may be covalently linked to the surface of the particle.

In some embodiments, the targeting agents of the present disclosure may be associated with a surface of a particle through a linker. In some embodiments, the linker is covalently coupled to a surface of a particle and to the targeting agent (e.g., linker 12 illustrated in FIG. 1A). In some embodiments, the linker is covalently coupled to a phospholipid on a surface of a molecule. In some embodiments, the linker is a small molecule, such as polyethylene glycol (PEG). In some embodiments, the linker can prolong the circulation of particles by stabilizing them against opsonization.

In some embodiments, the targeting agents of the present disclosure target adipose stromal cells. The targeting agents of the present disclosure may target adipose stromal cells in various manners. For instance, in some embodiments, the targeting agents of the present disclosure target an epitope on the adipose stromal cells. In some embodiments, the epitope includes a cleavage product of decorin. In some embodiments, the cleavage product of decorin is a decorin lacking the glycanation site (ΔDCN).

In more specific embodiments, the targeting agents of the present disclosure target a receptor on adipose stromal cells, such as a receptor that is expressed in high amounts on the plasma membrane of adipose stromal cells. In some of such embodiments, the delivery of the active agents into the adipose stromal cells occurs by receptor-mediated endocytosis.

### Delivery Agent Forms

The delivery agents of the present disclosure may be in various forms. For instance, in some embodiments, the delivery agents of the present disclosure are embedded within hydrogels. In some embodiments, the hydrogels include a network of hydrophilic polymers. In some embodiments, the hydrophilic polymers include, without limitation, polyethylene oxide, polyvinylpyrrolidone, polyethylenimine, polyethylene glycol, polyvinyl alcohol, and combinations thereof.

In some embodiments, the delivery agents of the present disclosure are associated with various devices. In some embodiments, the devices include, without limitation, microneedles, transdermal devices, iontopherosis patches, patches, and combinations thereof.

### Adipose Stromal Cells

In some embodiments, the methods and delivery agents of the present disclosure may target various types of adipose stromal cells. For instance, in some embodiments, the adipose stromal cells may include adipose stromal stem cells. In some embodiments, the adipose stromal cells include adipose stromal progenitor cells. In some embodiments, the adipose stromal cells include adipose stromal stem cells and adipose stromal progenitor cells.

The adipose stromal cells of the present disclosure may also be associated with various types of cells. For instance, in some embodiments, the adipose stromal cells may be associated with fat cells that include, without limitation, stem cells, progenitor cells, brown adipocyte cells, white adipocyte cells, brown-like/beige adipocyte cells, and combinations thereof.

In various embodiments, adipose stromal cells may be targeted in vitro or in vivo. Moreover, in some embodiments, the adipose stromal cells may be a component of a tissue. In some embodiments, the tissue includes, without limitation, white adipose tissue, brown adipose tissue, beige adipose tissue, and combinations thereof. In some embodiments, the tissue is white adipose tissue. In some embodiments, the tissue is brown adipose tissue.

Association of Delivery Agents with Adipose Stromal Cells

Various methods may be utilized to associate delivery agents with adipose stromal cells. For instance, in some embodiments, the association occurs in vitro. In some embodiments, the association occurs in vivo in a subject, such as an obese subject.

The delivery agents of the present disclosure may be in various forms in association with adipose stromal cells. For instance, in some embodiments, the associating occurs while the delivery agents are embedded within hydrogels, microneedles, or other transdermal devices.

In some embodiments, the associating occurs by administering the delivery agent to the subject. In some embodiments, the administration occurs by intravenous administration. In some embodiments, the association occurs by subcutaneous administration (e.g., subcutaneous injection). In some embodiments, the association occurs by transdermal administration. In some embodiments, the association occurs by topical administration. In some embodiments, the association occurs by intra-arterial administration. In some embodiments, the association occurs by intra-arterial administration.

In some embodiments, the administration of the delivery agent can have various therapeutic effects on the subject. For instance, in some embodiments, the administration of the delivery agent treats or prevents obesity in the subject. In some embodiments, the administration of the delivery agent treats or prevents a disorder or disease in the subject. In some embodiments, the disorder or disease is associated with obesity. In some embodiments, the disorder or disease includes, without limitation, metabolic syndromes, diabetes, type 2 diabetes, cardiovascular diseases, hypertension, coronary heart diseases, insulin resistance, dyslipidemia, cancer, osteoarthritis, rheumatoid arthritis, aging, wrinkles, alopecia, liver failure, multiple sclerosis, obesity, and combinations thereof.

In some embodiments, the administration of the delivery agents of the present disclosure occurs by subcutaneous or transdermal administration. In some embodiments, the subcutaneous or transdermal administration maximizes fat loss in a subject. In some embodiments, the subcutaneous or transdermal administration maximizes changes in the fat content of a desired body area.

Without being bound by theory, the delivery agents of the present disclosure can have various therapeutic effects on a subject through various mechanisms. For instance, in some embodiments, the administration of the delivery agents of the present disclosure decreases body weight in the subject. In some embodiments, the administration of the delivery agents of the present disclosure increases insulin sensitivity in the subject. In some embodiments, the administration of the delivery agents of the present disclosure decreases inflammation in the subject. In some embodiments, the administration of the delivery agents of the present disclosure improve blood lipid profile in the subject. In some embodiments, the administration of the delivery agents of the present disclosure decreases risk of cardiovascular disease in the subject. In some embodiments, the administration of the delivery agents of the present disclosure increases energy expenditure in the subject.

In some embodiments, the administration of the delivery agents of the present disclosure reduces fasting blood glucose levels in a subject. In some embodiments, the administration of the delivery agents of the present disclosure reduces fasting blood glucose levels in the subject by at least 20%. In some embodiments, the administration of the delivery agents of the present disclosure reduces fasting blood glucose levels in the subject by at least 26%.

In some embodiments, the administration of the delivery agents of the present disclosure reduces fasting blood insulin levels in a subject. In some embodiments, the administration of the delivery agents of the present disclosure reduces fasting blood insulin levels in the subject by at least 50%. In some embodiments, the administration of the delivery agents of the present disclosure reduces fasting blood insulin levels in the subject by at least 60%. Improve insulin sensitivity by at least 50%.

In some embodiments, the administration of the delivery agents of the present disclosure reduces inflammation in a subject. In some embodiments, the administration of the delivery agents of the present disclosure reduces inflammation in a subject by lowering plasma concentrations of various inflammatory markers, such as TNF-α, IL-6, IFN-γ and MCP-1.

In some embodiments, the administration of the delivery agents of the present disclosure reduces total blood cholesterol concentrations in a subject. In some embodiments, the administration of the delivery agents of the present disclosure reduces blood HDL concentrations in a subject. In some embodiments, the administration of the delivery agents of the present disclosure reduces blood LDL concentrations in a subject.

In more specific embodiments, the administration of the delivery agents of the present disclosure treats or prevents obesity in a subject. In some embodiments, the administration of the delivery agents of the present disclosure treats or prevents obesity in a subject by decreasing fat storage in the subject. In some embodiments, the administration of the delivery agents of the present disclosure decrease fat storage in the subject by conversion of white adipose tissue to brown adipose tissue, brown-like adipose tissue, beige adipose tissue, or combinations of such tissues in the subject. The increase in brown adipose tissue and brown-like adipose tissue can then result in more energy expenditure and less storage of fat throughout a subject's body.

In some embodiments, the administration of the delivery agents of the present disclosure treat or prevent obesity in a subject by conversion of adipose stromal cells in brown adipose tissues into brown adipocytes. In some embodiments, the administration of the delivery agents of the present disclosure treat or prevent obesity in a subject by increasing the activities and amounts of brown adipose tissue in the subject.

In some embodiments, the administration of the delivery agents of the present disclosure treat or prevent obesity in a subject by conversion of adipose stromal cells in beige adipose tissues into brown adipocytes. In some embodiments, the administration of the delivery agents of the present disclosure treat or prevent obesity in a subject by increasing the activities and amounts of beige adipose tissue in the subject.

Without being bound by theory, the administration of the delivery agents of the present disclosure can convert white adipose tissue to brown adipose tissue or brown-like adipose tissue in a subject through various mechanisms. For instance, in some embodiments, the conversion occurs by inducing mRNA expression of browning markers in a white adipose tissue, such as UCP1, PRDM16, PGC1α, CD 137, and PPARγ. In some embodiments, the conversion occurs by suppressing mRNA expression of white specific markers in the white adipose tissue, such as IGFBP3 mRNA expression. Examples of such modes of action are illustrated in FIGS. 1D-1F.

### Applications and Advantages

The methods and delivery agents of the present disclosure have numerous advantages. For instance, in some embodiments, the delivery agents of the present disclosure protect active agents by encapsulating the active agents in a particle and targeting the active agents to desired adipose stromal cells. Such a mode of delivery that combines targeted delivery and protected delivery helps reduce or mitigate the pharmacologic problems associated with various active agents (e.g., resveratrol), including limited solubility, limited stability, limited bioactivities, and limited ability to reach desired adipose stromal cells. Such a mode of delivery also increases the uptake of active agents by the desired adipose stromal cells.

Moreover, in some embodiments, the delivery agents of the present disclosure can be utilized to carry multiple active agents to adipose stromal cells. The delivery agents of the present disclosure can also be utilized to increase molecular stability, solubility, and bioavailability. The delivery agents of the present disclosure can also be utilized to decrease molecular toxicity. In addition, the delivery agents of the present disclosure can be utilized to prolong the circulation and sustained release of the active agents of the present disclosure.

As such, the methods and delivery agents of the present disclosure can have numerous applications. For instance, in some embodiments, the delivery agents of the present disclosure can be utilized as dietary pharmaceuticals for weight loss and weight management. In some embodiments, the delivery agents and methods of the present disclosure can be utilized to help better control the weight of numerous subjects without the use of invasive surgical procedures and with much more success than lifestyle alone.

### Additional Embodiments

Reference will now be made to more specific embodiments of the present disclosure and experimental results that provide support for such embodiments. However, applicants note that the disclosure herein is for illustrative purposes only and is not intended to limit the scope of the claimed subject matter in any way.

### Example 1. Resveratrol Liposomes and Lipid Nanocarriers: Comparison of Characteristics, Including Browning of White Adipocytes

Trans-resveratrol (R) has a potential to increase energy expenditure via inducing browning in white adipose tissue. However, its low levels of aqueous solubility, stability, and poor bioavailability limit its application. Applicants have successfully synthesized biocompatible and biodegradable R encapsulated lipid nanocarriers (Rnano), and R encapsulated liposomes (R-lipo). The mean particle size of Rnano and R-lipo were around 140 nm and 110 nm, respectively, and their polydispersity index values were less than 0.2. Nanoencapsulation significantly increased aqueous solubility and enhanced chemical stability of R, especially at 37° C. R-lipo had higher physical and chemical stability than Rnano while Rnano had more prolonged release than R-lipo. Both Rnano and R-lipo increased cellular R content in 3T3-L1 cells. Both Rnano and R-lipo dose-dependently induced uncoupling protein 1 (UCP1) mRNA expression, and decreased white specific marker insulin growth factor binding protein 3 expression under isoproterenol (ISO)-stimulated conditions. At the low dose (5 μM), nanoencapsulated compared to native R enhanced UCP1 and beige marker CD137 expression under ISO-stimulated conditions. Compared to Rnano, R-lipo had better biological activity, possibly due to its higher physical and chemical stability at the room and body temperature. Taken together, Applicants' results demonstrate that nanoencapsulation increased R's aqueous solubility and stability, which were associated with enhanced browning of white adipocytes. Even though both R-lipo and Rnano increased R's browning activities, their differential characteristics need to be considered in obesity treatment.

Obesity remains to be the major public health issue in the United States and worldwide, paralleled by rising rates of co-morbidities such as metabolic syndrome, diabetes, coronary heart disease, and certain types of cancer. Two different adipose tissues are found in mammals: white adipose tissue (WAT), which is responsible for energy storage; and brown adipose tissue (BAT), which is responsible for thermogenic energy expenditure. BAT has been positively associated with energy expenditure and negatively associated with adiposity in animal models and humans. Uncoupling protein 1 (UCP1) found in the inner mitochondrial membrane of brown adipocytes in the BAT can dissipate the proton electrochemical gradient generated from oxidative phosphorylation in the form of heat. Emerging data have demonstrated that UCP1 is expressed not only in classical brown adipocytes but also in “brown-like” or beige adipocytes within WAT upon stimulations, such as a chronic cold challenge or pharmacological or bioactive compounds. To increase adipocyte UCP1 expression and induce WAT “browning” might result in enhanced thermogenic and fat-burning activities, which subsequently lead to weight loss.

Trans-resveratrol (3,5,4′-trihydroxy-trans-stilbene, R) is a polyphenolic compound, abundant in the skin of grapes and red wine. Many in vitro studies have demonstrated that R at concentrations between 10 to 100 μM exhibits anti-obesity activities by modulating adipocyte differentiation, lipolysis, fatty acid oxidation, and mitochondria biogenesis and activities. R activates NAD-dependent deacetylase sirtuin 1 (SIRT1), which can deacetylate peroxisome-proliferator-activated receptor γ (PPARγ). This modification is essential for enhancing PPARγ-binding activity, recruiting transcription factor PR-domain-containing 16 (PRDM16) to PPARγ, and activating PPARγ co-activator 1α (PGC1α), which subsequently enhance UCP1 expression and initiate browning of WAT. Native R (at 10 μM) enhanced mRNA expression of UCP1 and beige marker CD137 and Tmem26 during brown-like differentiation of primary stromal stem/progenitor cells derived from inguinal WAT (iWAT) of CD1 mice. Consistently, in vivo feeding of R promoted the appearance of multilocular adipocytes and increased UCP1 expression in iWAT in the mice fed with a high fat diet. These results suggest that browning WAT may be a new anti-obesity target for R.

Human studies indicate that R can maintain metabolic health, but the evidence is inconclusive. The major problems are R's low aqueous solubility and bioavailability, and high metabolism in humans. The solubility of R in water and physiological fluid is very low (i.e., less than 0.1 mg/mL). When orally administering around 0.3 mg/kg body weight of R to healthy adult males, blood peak concentrations of R appeared at 0.5 hr, and the peak plasma concentrations were less than 1 μM. Even when R was given a single dose of 5 g, the peak plasma concentrations were still less than 10 μM. Moreover, R stability is further reduced by various metabolic transformations, including methylation, glucuronidation and others, primarily in the liver in vivo.

Nanoencapsulation has been proved effective in increasing aqueous solubility, chemical stability, and bioavailability of many bioactive compounds in combating obesity and associated metabolic disorders. See Bonechi et al., PLoS One, 7 (2012) e41438. In addition, recent animal and human studies indicate that encapsulating R into nanocarriers can increase R's aqueous solubility, protect R from metabolic degradation, and enhance its transport across the plasma membrane, with ultimately augmented absorption and bioavailability. See Singh et al., Drug delivery, 22 (2015) 522-530.

In this Example, Applicants synthesized R encapsulated lipid nanocarriers (Rnano) and R encapsulated liposomes (R-lipo), two biodegradable and biocompatible nanocarrier delivery systems, and compared their physical and physicochemical characteristics and browning activities with native R in 3T3-L1 white adipocytes.

### Example 1.1. Chemicals and Reagents

R was purchased from Cayman Chemical Co. (+)-Alpha (α)-tocopherol acetate (αTA), cholesterol, dexamethasone (Dex), 3-isobutyl-L-methylxanthine (IBMX), insulin, isoproterenol (ISO) and rosiglitazone (Rosi) were purchased from Sigma-Aldrich Chemical Co. Soy L-a-phosphatidylcholine (PC) was purchased from Avanti Polar Lipids Inc. Kolliphor® HS15 was given as a gift from BASF Chemical. All organic solvents were high-performance liquid chromatography (HPLC) grade.

### Example 1.2. Preparation of Rnano and R-Lipo

Rnano was prepared using a mixture containing 1 mg of R, 70 mg of soy PC, 17.6 mg of Kolliphor® HS15 and 18 mg of αTA. The mixture was dissolved in ethanol and completely dried under nitrogen gas. After suspending the mixture with 76° C. deionized water, the suspension was homogenized for 1 min followed by sonication for 1 min. The Rnano tube was put on ice immediately. After ultrafiltration to remove free R, the Rnano was resuspended into 1* phosphate-buffered saline (1×PBS). R-lipo was prepared using 1 mg of R, 20 mg of soy PC and 2 mg of cholesterol by a film dispersion method followed by a membrane extrusion method. The void nanocarriers (V-nano) and void liposomes (V-lipo) were prepared using the above methods without adding R.

### Example 1.3. Particle Size, Zeta Potential, and Morphology

The particle size and polydispersity index (PI) values of Rnano, R-lipo and their void counterparts were measured using a Brookhaven BI-MAS particle size analyzer, and the zeta potential was measured using a Zeta PALS analyzer. The morphology and size of nanocarriers and liposomes were determined using a 200 kV Hitachi H-8100 transmission electron microscope (TEM) as described.

### Example 1.4. Physical and Chemical Stability

The freshly made Rnano and R-lipo were aliquoted into transparent or black tubes and stored at 4° C., 22° C., and 37° C. for 7 days, and their physical and chemical stability was measured during this period. The mean particle size, PI and zeta potential, were measured every 2 hours for the first 10 hours, and every 24 hr for 7 days. The chemical stability of Rnano, R-lipo and native R was measured using the HPLC system every day for 7 days.

### Example 1.5. In Vitro Release Studies

Before the in vitro release study, the stability of Rnano, R-lipo and native R in the dissolution medium were measured at 37° C. for 24 hr. The dissolution medium was composed of 1×PBS and methanol (80:20, v/v). The in vitro release behaviors of Rnano, R-lipo and native R containing 0.5 mg of R were performed in the dissolution medium using a dialysis method as described. See Sun et al., Colloids Surf B Biointerfaces, 113 (2014) 15-24.

### Example 1.6. Cell Culture and Viability Studies

Murine 3T3-L1 fibroblasts purchased from ATCC were grown in Dulbecco's modified Eagle's medium (DMEM) containing 10% calf serum following a standard protocol, and cell viability was measured by the colorimetric 3-(4,5-dimethylthiazolyl-2)-2,5-diphenyltetrazolium bromide (MTT) assays.

### Example 1.7. Cellular R Content Studies

R content in 3T3-L1 cells were studied using the HPLC system. Quercetin was used as an internal standard. Total cellular R content was expressed as g of R per mg of protein.

### Example 1.8. Real Time-PCR

Total RNA was extracted, cDNA was synthesized, and real-time PCR was performed using an ABI 7300HT instrument.

### Example 1.9. Transfection and Reporter Gene Assays

3T3-L1 cells were transiently transfected with a peroxisome proliferator response element (PPRE)-driven luciferase reporter (PPRE-Luc) and P-galactosidase (P-gal) control plasmid with Lipofectamine 3000 and PLUS Reagent for 24 hr. The cells were then treated with nanoparticles and the controls for an additional 15-18 hr. The luciferase activities were measured by a Promega GloMax-Multi Detection System and normalized by the P-gal activities.

### Example 1.10. Statistical Analysis

Statistical analysis was performed using Statistical Package for the Social Sciences (SPSS) and SigmaPlot 13 (Systat Software, Inc). One-way ANOVA was performed followed by multiple comparison tests with Student-Newman-Keuls method or Holm-Sidak method to compare with controls. Each experiment was conducted independently at least three times; each measurement was performed in triplicates within each experiment. The level of significance was set at p<0.05.

### Example 1.11. Characteristics of Rnano and R-Lipo

Rnano and R-lipo were successfully synthesized. Traditional nanostructured lipid carriers (NLCs) as drug carriers usually contain a large amount of triglyceride. Applicants have successfully replaced triglyceride with αTA, consequently eliminating exogenous triglyceride and increasing the anti-oxidative capacity of nanocarriers, making them more functional and beneficial.

FIG. 2A shows that 1 mg of native R was hardly dissolved in 1 mL of 1×PBS and precipitated from the suspension immediately. However, 1 mg of nanoencapsulated R in both Rnano and R-lipo was dissolved in the same volume of 1×PBS, which had 25-fold higher aqueous solubility than native R. Both Rnano and R-lipo were translucent and opalescent (FIG. 2A). Several studies have demonstrated that entrapment of R in nanoparticles increase R's aqueous solubility and further bioavailability. See Wang et al., J Nutr Biochem, 25 (2014) 363-376. TEM images indicated that both Rnano and R-lipo were spherical (FIG. 2A). The average particle size of Rnano and R-lipo was around 140 nm and 110 nm, respectively (FIG. 2A).

Recently, research data indicate that tissue distribution of nanocarriers was size-dependent, and nanocarriers close to 100 nm was found to be delivered effectively into adipose tissues of obese mice. The PI values of Rnano and R-lipo were 0.084 and 0.140, respectively. The low PI values indicated a high level of size homogeneity of Rnano and R-lipo. Higher PI values (>0.3) indicate higher levels of heterogeneity.

Surface charges of nanocarriers play vital roles in cellular uptake, bio distribution, and bioavailability of nanocarriers. The Zeta potentials of freshly made Rnano and R-lipo were around −19 and −28 mV, respectively. Soy PC, the major surface component of Rnano and R-lipo, rendered negative charges on the surface of particles. In general, they can be dispersed stably due to the electric repulsion of negative charges among particles.

The encapsulation efficiency of Rnano and R-lipo was 96.5% and 96.0%, respectively; and R's loading capacity in Rnano and R-lipo was 28.5% and 25.3%, respectively. Since there is a high proportion of hydrophobic αTA on Rnano, Applicants predict that Rnano might have a monolayer of PC and Kolliphor® HS15 on the surface and a hydrophobic αTA core (FIG. 2A). The high encapsulation efficiency and loading capacity of Rnano were partially due to the hydrophobicity of αTA, which accommodates more R in the hydrophobic core. R-lipo might have multiple PC bilayers and a hydrophilic core. Due to the biphasic characteristics of PC, R can be embedded into the sections of hydrophobic fatty acid tails of PC bilayer. Multiple PC bilayers on liposomes also accommodate a good amount of R.

### Example 1.12. Physical and Chemical Stability

Storage of both Rnano and R-lipo at 4° C. for 4 days did not change their diameters and zeta potentials significantly. Furthermore, the PI values remained under 0.2, indicating relatively good homogeneity and physical stability (FIG. 2B). Storage of Rnano at room temperature (22° C.) for 4 days increased its diameters and PI values slightly (FIG. 2B). At 37° C., Rnano can only maintain its original size for 8 hr, and its PI values were gradually increased (FIG. 2B).

Two reasons may contribute to the poor physical stability of R-nano at 37° C. First, the hydrophobic lipid core of Rnano was composed of αTA. The melting point of αTA was around 25° C. (Sigma T3001), as indicated on the product sheet, and the solid core would turn to liquid resulting in the fragile and instable structure of Rnano.

Second, a high temperature may break the hydrogen bonds of Kolliphor® HS15, the surfactant incorporated on the surface of Rnano, leading to a reduced stability of Rnano at 37° C. The diameter and zeta potential of R-lipo did not change significantly at 22° C. and 37° C. for 7 days, and the PI values remained under 0.2 (FIG. 2B), indicating its higher levels of physical stability and homogeneity than Rnano. It should be emphasized that the cholesterol was incorporated into R-lipo to increase its physical stability. Even though R-lipo is more stable than Rnano, other characteristics and anti-obesity bioactivities should be considered and measured.

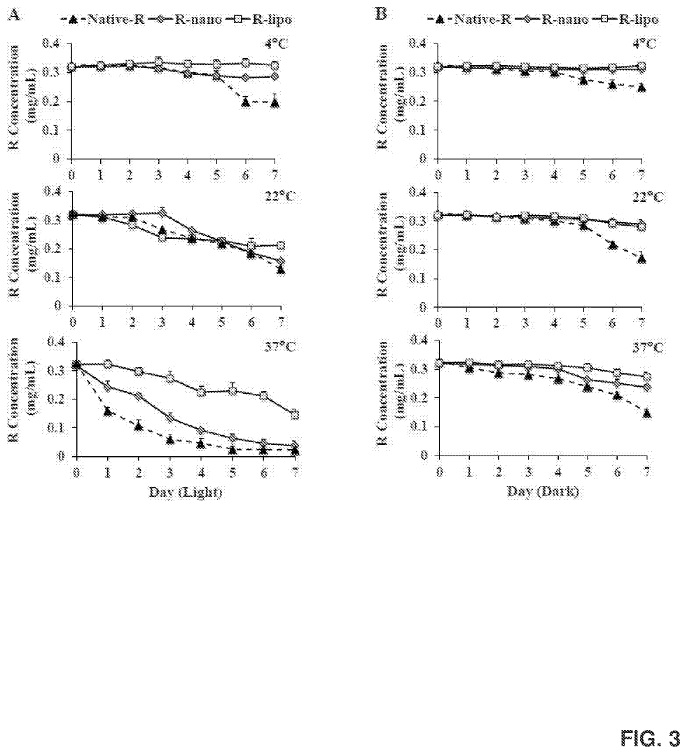

Rnano and R-lipo also enhanced the chemical stability of R that is sensitive to light. In a neutral pH 7.4 condition, native R was degraded by 40% at 4° C., 60% at 22° C. and 96% at 37° C. under the light after 7 days (FIG. 3A). Storage of native R in the dark decreased the R degradation rates to 17% at 4° C., 48% at 22° C., and 52% at 37° C. after 7 days (FIG. 3B). After incubation at 37° C. for 6 days under the light, R degradation rates were 90%, 80% and 20% in native R, Rnano, and R-lipo, respectively (FIG. 3A). Both lipo and nano structures protected R from degradation no matter under dark or light, and their protective capability was similar at 4° C. and 22° C. (FIG. 3).

Rnano might be solid at the room temperature because the melting temperatures of αTA (Sigma T3001), soy PC (Avanti 441601) and Kolliphor® HS15 are 25° C. and above. The hydrophobic R can be encapsulated into the solid core, which can enhance its stability and prolong its release. R-lipo had better protective capacity than Rnano at 37° C. The higher R degradation rate in Rnano could be partially due to lack of a solid phase of Rnano at 37° C. Additionally, many research studies have indicated the nanoencapsulation can increase R chemical stability via protecting it from light degradation.

### Example 1.13. Raman Spectroscopy Analysis

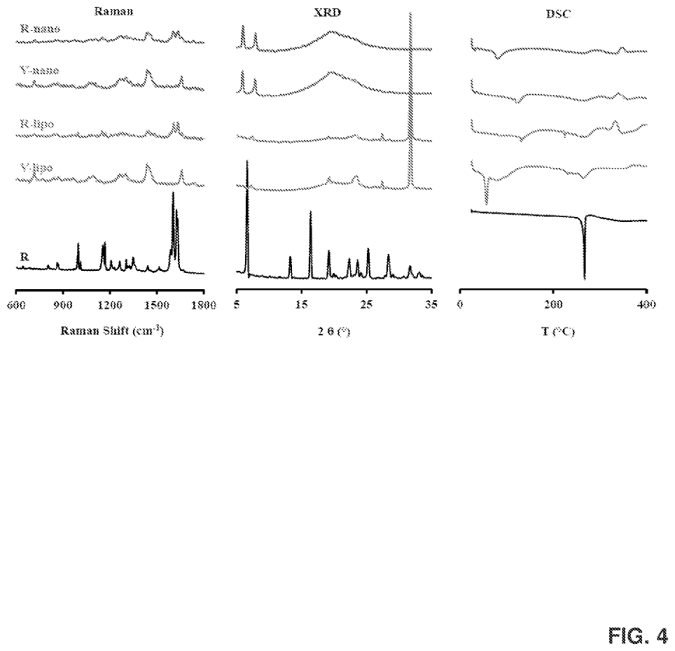

Raman spectroscopy is a proven method to quickly and effectively identify encapsulated active agents. Raman spectra of native R, Rnano, V-nano, R-lipo, and V-lipo are presented in FIG. 4. The spectrum of native R shows three major characteristic bands, including olefinic bands at 980-1022 cm⁻¹, C—O stretching at 1132-1188 cm⁻¹, and C—C aromatic double-bond stretching at 1570-1655 cm⁻¹, in addition to several other weak features that are consistent with previously identified bands for R. The Raman spectra of V-lipo and V-nano are very similar, dominated by characteristic peaks of PC, the common component of these two nanocarriers. Since the signatures for native R and V-nano/V-lipo have no overlap, it becomes unambiguous to differentiate the compositions in the encapsulated particles and void nanocarriers. As observed from the spectra, the major characteristic peaks of native R were present in Rnano and R-lipo, but absent in V-nano and V-lipo, while those peaks of V-nano and V-lipo were present in their corresponding Rnano and R-lipo, but not in native R. These clearly indicate the encapsulation of R into Rnano and R-lipo.

### Example 1.14. X-Ray Diffraction (XRD) Analysis

The XRD analysis can effectively distinguish between the crystalline and amorphous phases of Rnano and R-lipo. The crystallinity of nanocarriers is desirable because the solubility and dissolution rate of Rnano and R-lipo in the R delivery process can be significantly affected by the degree of crystallinity. The XRD patterns of native R, R-nano, R-lipo and the corresponding control forms and physical mixtures were shown in FIG. 4. The diffractogram of native R exhibited intense peaks between 5° and 35°, indicating the native R in a highly crystalline form. In the lyophilized nanocarriers, the sharp peaks from the crystalline native R were absent, suggesting less crystallinity or more amorphous state after R molecules were loaded in the soy PC shell to form an amorphous complex.

Although the Raman spectra of V-lipo and V-nano are very similar, their XRD patterns are dramatically different, suggesting their different composition and structures, as two different nanocarriers. With the absence of R crystalline peaks, XRD patterns of R-lipo and Rnano present the features of V-lipo and V-nano, respectively.

### Example 1.15. Differential Scanning Calorimetry (DSC) Analysis

The DSC curves of native R, Rnano, R-lipo and the corresponding control forms were presented in FIG. 4, as a second method to examine the crystallinity. The difference between V-lipo and V-nano again was notified. The DSC thermogram of native R showed the characteristic endothermic peak at 267° C., corresponding to its melting temperature. However, R's endothermic peak was disappeared in the DSC scans of Rnano and R-lipo, suggesting its amorphous phase in these nanocarriers. Interestingly, the DSC curves of R-lipo and V-lipo and those of Rnano and V-nano exhibit different features. This difference strongly suggests that R molecules were dispersed into the two nanocarriers, resulting in the amorphous phase. This conclusion is reinforced by the XRD measurements in which only the characteristic crystalline peaks of the V-nano or V-lipo were observed. All three physicochemical analyses confirm that R drug amorphization occurred in both R-nano and R-lipo nanocarriers.

### Example 1.16. In Vitro Release

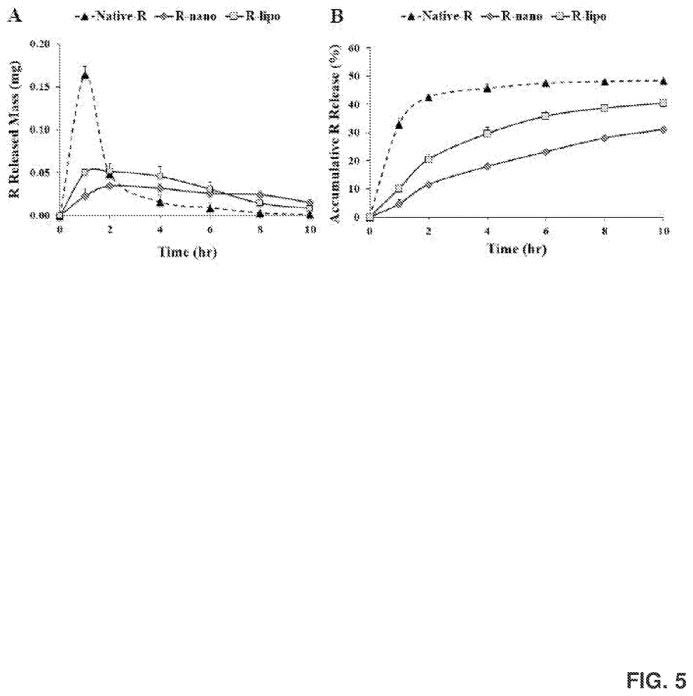

Hydrophobic native R has a low level of aqueous solubility. Methanol was added to make the dissolution medium to dissolve released native R. Different methanol contents had been tested before conducting the release study to ensure to use the minimal amount of methanol. The dissolution medium containing 20% methanol was the optimal formula, which could dissolve almost all released native R without destroying nanostructures (data not shown). The release study was conducted in the dark to prevent light-induced R degradation. To minimize the effect of R stability, the dissolution medium was changed completely each hour for the first 2 hr, and every 2 hr for the rest 8 hr. Native R showed a burst release phenomenon, while Rnano and R-lipo exhibited a sustained release behavior (FIG. 5).

After 1 hr, about 0.17 mg, 0.05 mg, and 0.02 mg of R was released from dialysis bags containing native R, R-lipo, and Rnano, respectively (FIG. 5A). At the 2-4 hr period, about 0.02 mg, 0.05 mg and 0.04 mg of R was released from dialysis bags containing native R, R-lipo, and Rnano, respectively (FIG. 5A). After dialysis for 2 hr, the accumulative released R mass from the native R dialysis bag reached a plateau (FIG. 5B), while for R-lipo and Rnano, R was continuously released in the 10 hr period. Furthermore, R-lipo released more R than Rnano (FIG. 5B). Hydrophobic compounds like R might release faster from the membrane PC bilayers of liposomes than from the hydrophobic core of nanocarriers. The data indicate R is probably distributed in the PC bilayers of R-lipo, and in the hydrophobic core of Rnano.

### Example 1.17. Cytotoxicity

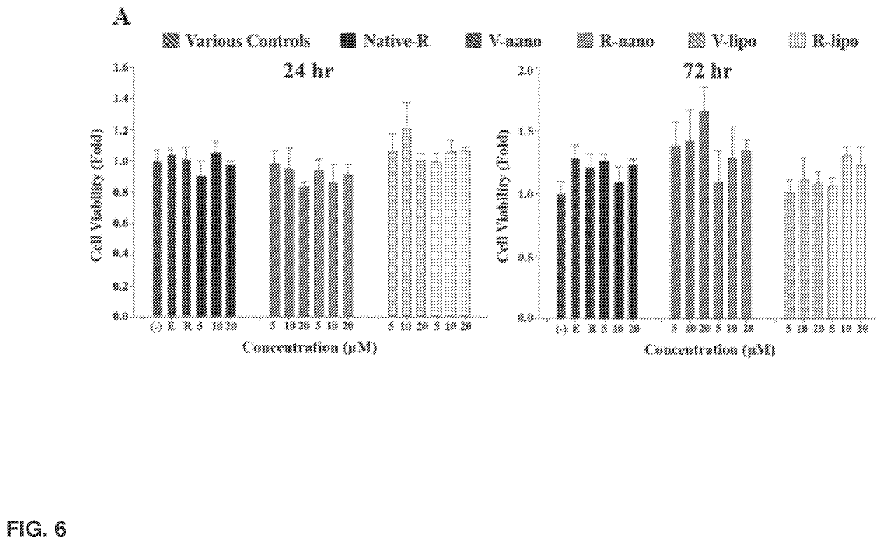

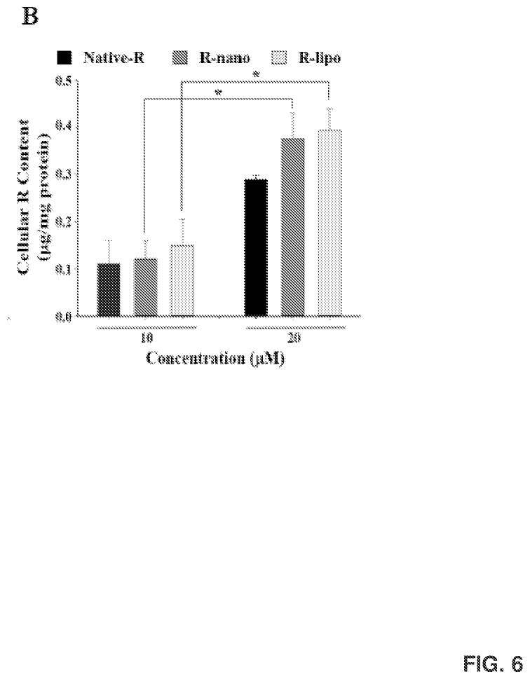

None of the forms of R at tested doses negatively affected cell viability in 3T3-L1 cells after 24 or 72 hr (FIG. 6A). The results suggest that two biocompatible and biodegradable nanostructures are safe to use, at least in 3T3-L1 cells.

### Example 1.18. Cellular R Content

To investigate cellular bioavailability, Applicants measured the R content in 3T3-L1 cells (FIG. 6B). The cellular R content was dose-dependently increased by all treatments. As compared to native R, Rnano and R-lipo only slightly increased R content at 10 μM; but increased R content by more than 20% and 25% at 20 μM, respectively. Due to the instability of native R at 37° C., 3T3-L1 cells were only treated for 4 hr in this experiment. Considering only 4 hr treatment, the increase in cellular R content by Rnano and R-lipo is significant.

### Example 1.19. Activation of PPAR Responsive Reporter

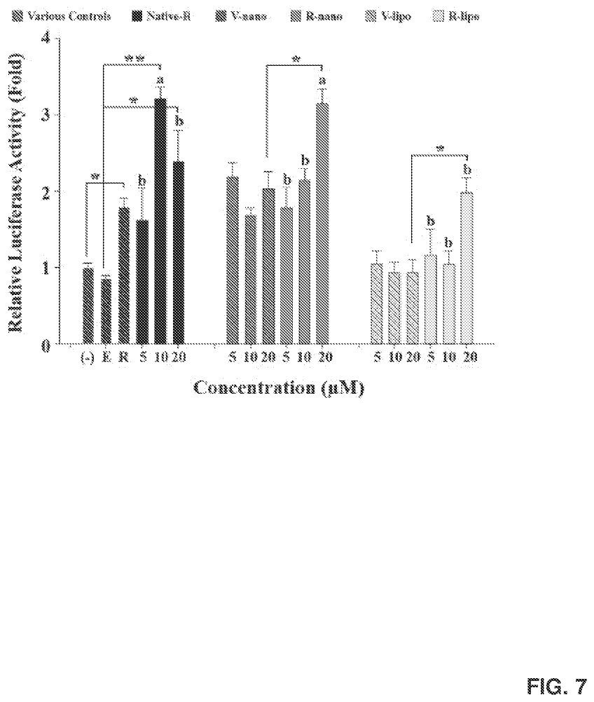

It has been reported that R (native form) activates PPARγ, which heterodimerizes with retinoid X receptor (RXR) to bind to PPRE sites in the promoter regions to transactivate target genes, including UCP1. All forms of R significantly activated PPRE-Luc compared to their controls (p<0.05). No significant differences were detected among the various forms of R (FIG. 7). R-lipo and Rnano had similar activation abilities compared to native R (FIG. 7), a property that is associated with browning activities of rosiglitazone. The results suggest that encapsulation did not change the biological capability of R in activating PPARγ.

### Example 1.20. Browning Activities

Applicants further studied browning effects of native R, Rnano, and R-lipo in differentiating 3T3-L1 white adipocytes, a commonly used cell model to study browning. The hallmark of beige adipocytes is induced thermogenesis in response to stimuli, such as P-adrenergic agonist ISO. Applicants investigated the effects of native R, R-lipo, and Rnano on the gene expression of markers of brown and white adipocytes, and mitochondrial biogenesis under either basal (non-stimulated) or ISO-stimulated conditions.

### Example 1.21. Effects on mRNA Expression of Browning Markers

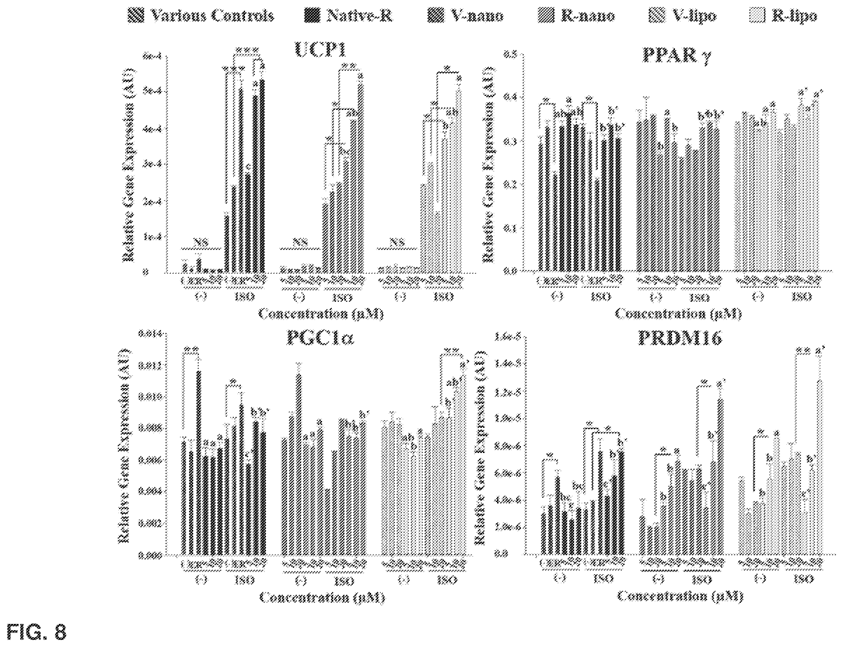

Neither rosiglitazone (Rosi) (positive control) nor any form of R significantly induced UCP1 mRNA expression at basal conditions. However, upon ISO stimulation, all forms of R significantly and dose-dependently induced UCP1 mRNA expression compared to their controls (p<0.05), similar to rosiglitazone (Rosi) (p<0.05). When compared among various forms of R, there were no significant differences between R-lipo and Rnano. However, at 5 μM (the low dose), R-lipo induced a higher UCP1 expression than native R under ISO stimulated conditions (p<0.05) (FIG. 8).

PPARγ, PGC1α, and PRDM16 are known core regulators of browning and UCP1 mRNA expression. No forms of R affect PPARγ mRNA under the basal condition, in contrast to rosiglitazone (Rosi), which suppressed PPARγ mRNA; R-lipo induced higher PPARγ mRNA levels than Rnano and native R (p<0.05) (FIG. 8).

Under basal conditions, rosiglitazone (Rosi) significantly induced PGC1a mRNA expression (p<0.05). However, various forms of R did not induce significant changes in PGC1a expression at all tested doses. Under ISO stimulated conditions, R-lipo significantly induced PGC1a mRNA expression at 20 μM compared to its control (p<0.05) and to a level that is higher than R-nano and native R at the same dose (p<0.05). At 5 μM, both R-lipo and Rnano induced higher PGC1a mRNA than native R (p<0.05) (FIG. 8).

rosiglitazone (Rosi) significantly induced PRDM16 mRNA expression under both conditions (p<0.05). Under the basal conditions, native R did not change PRDM16 mRNA expression. In contrast, both R-lipo and Rnano dose-dependently increased PRDM16 mRNA expression, reaching significance at 20 μM (p<0.05). Under ISO stimulated conditions, all forms of R similarly and dose-dependently increased PRDM16 mRNA expression, reaching significance at 20 μM compared to the controls (p<0.05) (FIG. 8).

### Example 1.22. Effects on mRNA Expression of White and Beige Markers

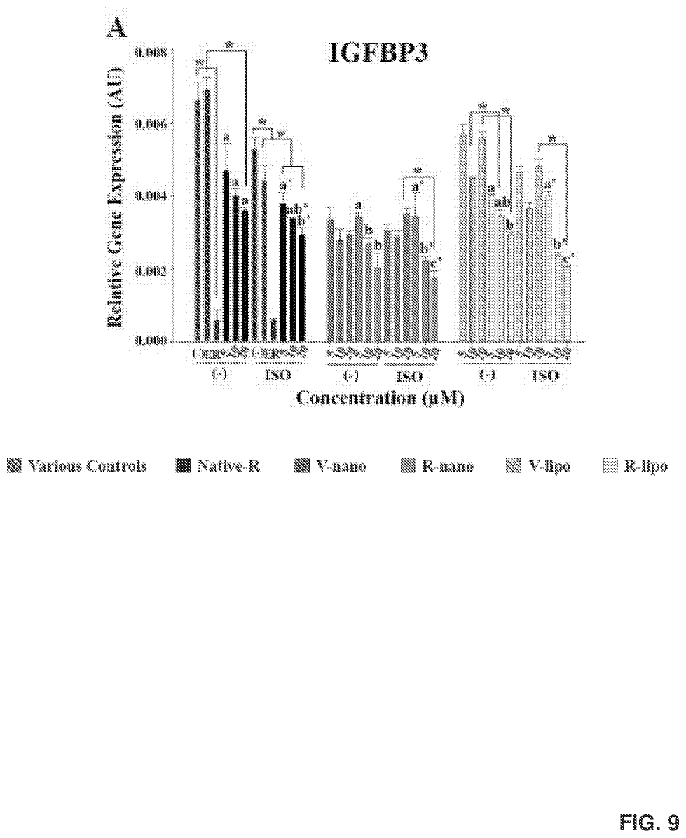

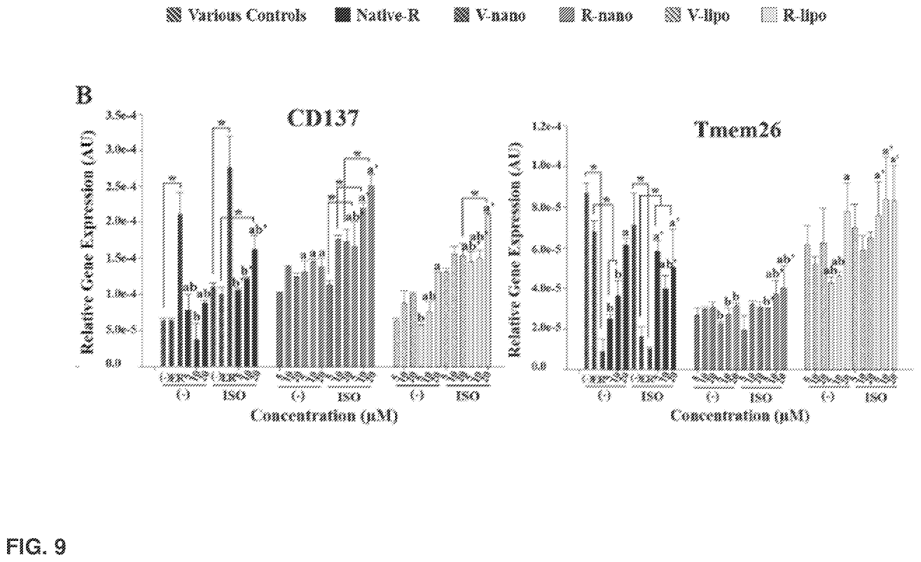

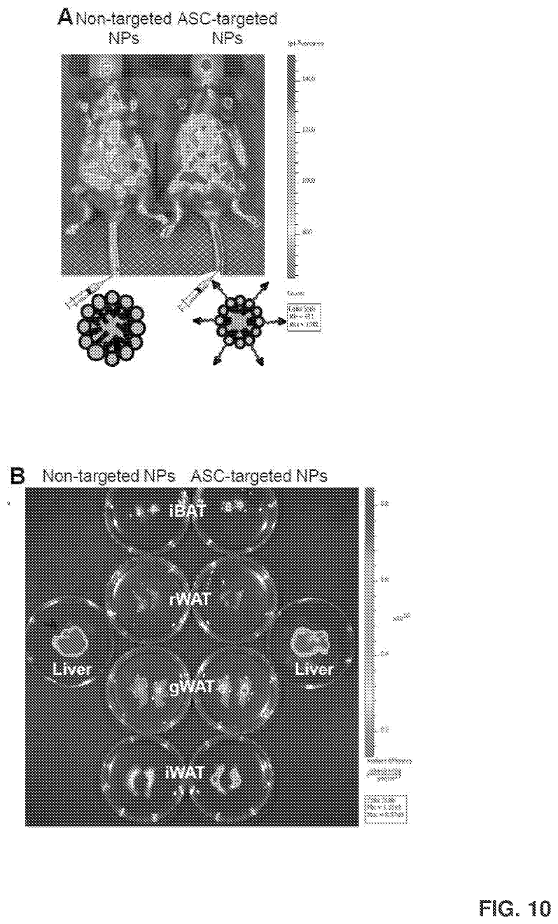

Next, Applicants examined various forms of R on white and beige marker mRNA expression during the browning of 3T3-L1 adipocytes. Insulin-like growth factor-binding protein 3 (IGFBP3) was identified as a white adipocyte specific marker by a transcriptome analysis of brown versus white adipocyte gene expression. rosiglitazone (Rosi) significantly decreased IGFBP3 mRNA expression (p<0.05) under both conditions, consistent with a previous report. Native R also dose-dependently decreased IGFBP3 expression compared to the controls under both conditions (p<0.05 at 20 μM under basal and p<0.05 at all tested doses under ISO stimulated conditions). Comparing to native R, both R-lipo and Rnano further decreased IGFBF3 mRNA expression at 20 μM at both conditions (p<0.05) (FIG. 9A).

CD137 and Tmem26 have been identified as beige specific markers. rosiglitazone (Rosi)significantly increased CD137 mRNA expression under both conditions (p<0.05). Various forms of R did not affect CD137 mRNA expression compared to the control under basal conditions. However, all forms of R dose-dependently increased CD137 mRNA expression under ISO stimulated conditions (p<0.05 for native R and R-lipo at 20 μM and p<0.05 for all tested doses for Rnano) (FIG. 9B).

Rosiglitazone (Rosi) significantly decreased Tmem26 mRNA expression under both conditions (p<0.05). Native R decreased Tmem26 mRNA expression under basal conditions (p<0.05 at 5 and 10 μM). In contrast, native R increased Tmem26 expression under ISO stimulation (p<0.05 at all tested doses). There were no differences in Tmem26 mRNA expression between R-lipo and Rnano and their controls under both conditions (FIG. 9B).

Using differentiating 3T3-L1 white adipocytes coupled with ISO-induced thermogenic activation, Applicants demonstrate, for the first time, that various forms of R enhanced ISO-induced mRNA expression of UCP1 and other browning markers, such as PRDM16 and PGC1α. In addition, various forms of R enhanced beige marker CD 137 mRNA expression but suppressed white specific marker IGFBP3 mRNA expression.

For the first time, Applicants demonstrated that various R suppressed IGFBP3 and Rnano and R-lipo had better suppression than native R at 20 μM under ISO-stimulated conditions. Taken together, R-induced browning may contribute to the beneficial effects of R for obesity and associated metabolic dysfunction.

Compared to Rnano, R-lipo induced significantly higher levels of UCP1 mRNA than native R when both were used at 5 μM (FIG. 8). Moreover, R-lipo induced higher levels of other browning marker, PGC1a than Rnano and native R under either basal and/or ISO stimulated conditions (FIG. 8). The better browning activities of R-lipo may be due to its higher physical and chemical stability compared to Rnano and native R. Moreover, the better biological activities demonstrated by R-lipo at 5 μM is more physiologically relevant since this dose is within the physiologically achievable dose range of R for human consumption. Compared to native R, both R-lipo and Rnano had better suppression of IGFBP3, possibly due to improved overall bioavailability by nanoencapsulation.

### Example 1.23. Effects on mRNA Expression of Mitochondrial Biogenesis Markers

Rosiglitazone (Rosi) significantly increased Tfam, Nrf, Cox4a, and Uqcrh under either basal and/or ISO stimulated conditions (p<0.05). There were minimal differences among the three forms of R in any of the mitochondrial biogenesis markers in 3T3-L1 cells except for native R, which increased Cox4a mRNA at 20 μM under ISO stimulated conditions (p<0.05).

In contrast to rosiglitazone (Rosi), all three forms of R had minimal effects on mitochondrial biogenesis genes under both basal and ISO stimulated conditions, suggesting that rosiglitazone (Rosi) and R may induce browning of 3T3-L1 adipocytes via different molecular mechanisms. Lack of changes in mitochondrial biogenesis suggests that R may induce browning of 3T3-L1 by directly upregulating UCP1 expression whereas Rosi may increase mitochondrial biogenesis.

Applicants' results suggest that various forms of R may promote browning by activating PPARγ. Currently, Applicants cannot rule out the possibilities that, in addition to activating PPARγ responsive promoters, R-lipo and Rnano may activate other signaling pathways, such as SIRT1, AMPK, or PDE, to induce browning.

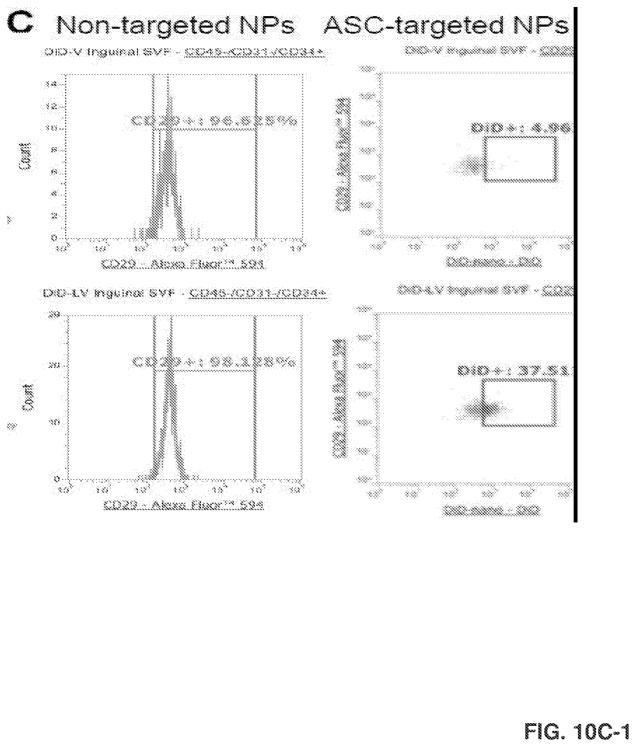

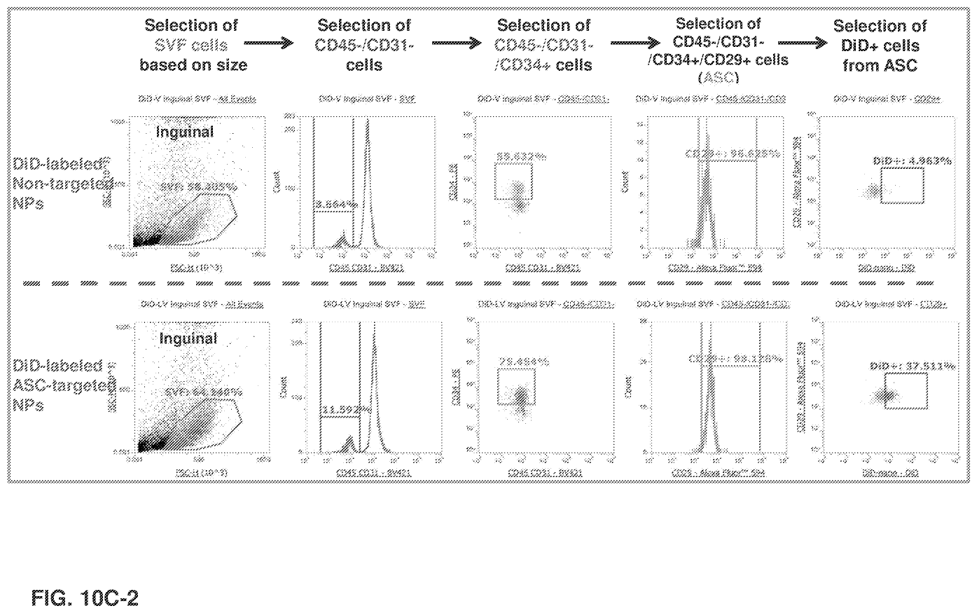

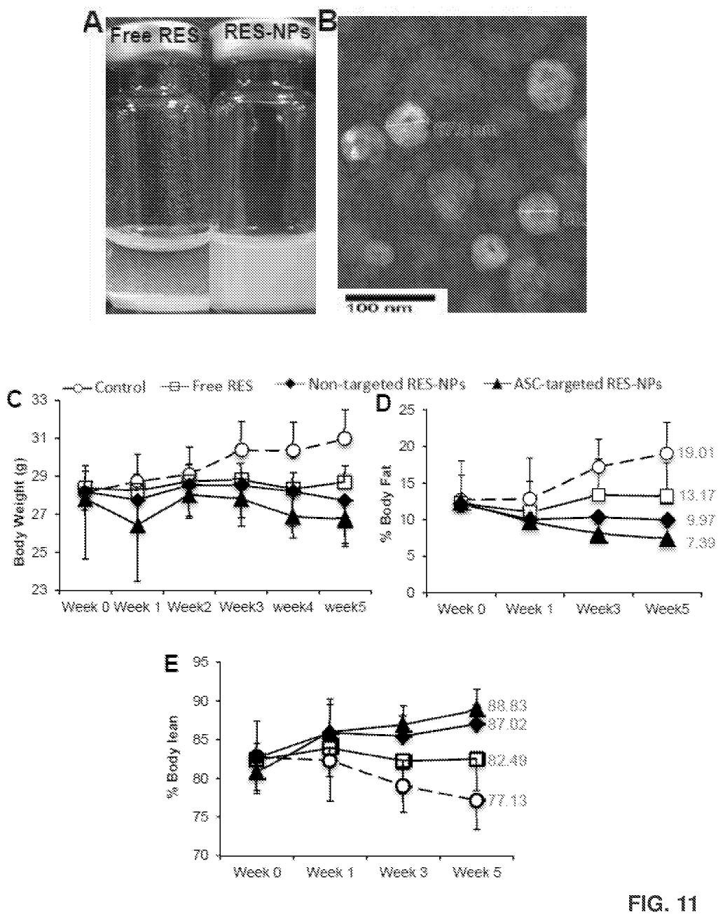

Additional experimental results are summarized in FIGS. 10 and 11. The results indicate that both R-lipo and Rnano have sustained release properties. The results also indicate that no cytotoxicity was observed at the used concentrations.

In sum, Applicants have successfully encapsulated R into nanocarriers and liposomes, which increased R aqueous solubility and stability. R-lipo showed higher physical and chemical stability but with less sustained release than Rnano. Both Rnano and R-lipo increased cellular R content in 3T3-L1 cells, which led to higher expression of UCP1, beige marker CD137 and other browning markers, and lower expression of white marker IGFBP3. Applicants' study demonstrates a novel strategy of using nanoencapsulation of R to achieve improved browning efficacy with minimal side effects.

### Example 2. Resveratrol Nanocarriers for Targeted Delivery of Resveratrol to Adipose Stromal Cells

The results in Example 1 demonstrated that R encapsulated nanoparticles (Rnano), consisting of soy phosphatidylcholine, alpha-tocopherol acetate, surfactant and R, significantly enhanced R aqueous solubility, chemical stability and sustained release pattern in vitro. Importantly, Rnano increased R cellular content in 3T3-L1 cells and dose-dependently induced beige marker UCP-1 and CD137 mRNA expression, which indicated the enhancement of beige adipocyte formation.

To achieve targeting specificity to ASCs in WAT and improve the browning efficacy of R, there is a considerable need to discover the specific target site and ligand of ASCs, and validate the target specificity of ligand-incorporated Rnano (L-Rnano) to ASCs in vitro and in vivo. Recently, decorin lacking the glycanation site (ΔDCN) has been identified as a functional receptor expressed on the surface of the mouse and human ASCs, and can be exploited as a specific molecular target site to facilitate ASC-based biomedical studies.

Therefore, Applicants fabricated a linear ASC-targeting peptide (GSWKYWFGEGGC) (SEQ ID NO: 2). Next, Applicants conjugated the linear ASC-targeting peptide with 1,2-distearoyl-sn-glycero-3-phosphoethanolamine-N-[maleimide(polyethylene glycol)-5000] (DSPE-PEG₅₀₀₀-Maleimide) to form DSPE-PEG₅₀₀₀-peptide via a thioether bond by a coupling reaction and successfully incorporated this DSPE-PEG₅₀₀₀-peptide on the surface of Rnano to synthesize ligand-coated Rnano (L-Rnano). Beyond the peptide's high binding affinity to ASCs, PEG5₀₀₀ on the surface of nanoparticles can maintain their integrity and stability by protecting them from degradation by enzymes and prolong the circulation of nanoparticles by stabilizing them against opsonization in vivo.

In this Example, Applicants sought to validate the high binding affinity and targeting specificity of L-Rnano to ΔDCN-transduced 3T3-L1 cells in vitro and WAT-derived ASCs identified as CD34⁺CD29⁺CD31⁻CD45⁻ cells from SVF in vivo. As expected, Applicants' data identified that L-Rnano, as compared to R and Rnano, enhanced the WAT browning effect in high fat diet (HFD)-induced obese C57B6LJ mice, subsequently resulting in high therapeutic anti-obesity efficacy, as well as improved obesity-related metabolic disorders.

### Example 2.1. Chemicals and Reagents

R was purchased from Cayman Chemical Co., (Ann Arbor, Mich., USA). (+)-α-tocopherol acetate (αTA), cholesterol, bovine serum albumins, and Type 1 collagenase were purchased from Sigma-Aldrich Chemical Co., (St. Louis, Mo., USA). Soy L-α-phosphatidylcholine (PC) and 1,2-dioleoyl-sn-glycero-3-phosphoethanolamine-N-(lissamine rhodamine B sulfonyl) (Rhoda) were purchased from Avanti Polar Lipids Inc. (Alabaster, Ala., USA). Kolliphor® HS15 (K) was given as a gift from BASF Chemical Co.

ASC-targeting peptides were synthesized by conventional peptide chemistry, cyclized via cysteines, purified to >95% purity by GenScript USA Inc. (Piscataway, N.J., USA). N-[(3-Maleimide-1-oxopropyl) aminopropyl polyethyleneglycol 5000-carbamyl]distearoylphosphatidyl-ethanolamine (SUNBRIGHT® DSPE-PEG₅₀₀₀-MAL) and N-(carbonyl-methoxypolyethyleneglycol 5000)-1,2-distearoyl-sn-glycero-3-phosphoethanolamine, sodium salt (SUNBRIGHT® DSPE-PEG₅₀₀₀) were purchased from NOF Corporation (Tokyo, Japan). 1,1′-Dioctadecyl-3, 3, 3′, 3′-tetramethylindodicarbocyanine, 4-chlorobenzenesulfonate Salt (DiD) was purchased from Thermo Fisher Scientific Co. (San Jose, Calif., USA). Type 1 collagenase was purchased from Worthington Biochemical Corp (Lakewood, N.J., USA).

### Example 2.2. Preparation of DSPE-PEG₅₀₀₀-Peptide Conjugate

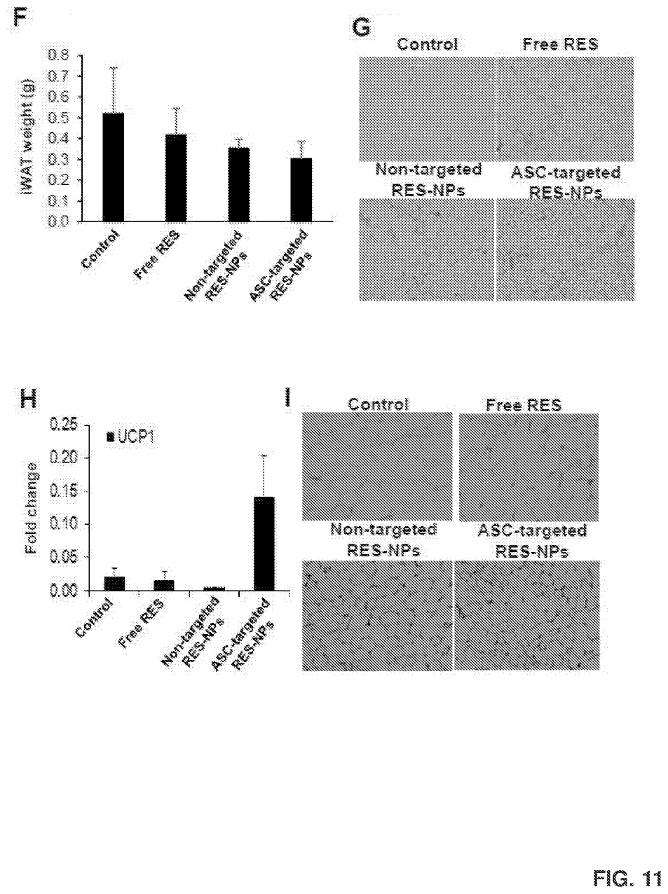

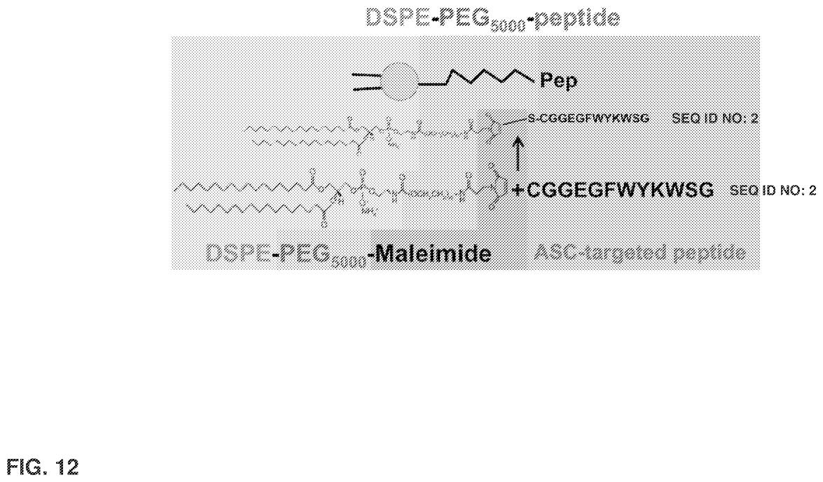

DSPE-PEG₅₀₀₀-peptide conjugate was synthesized from DSPE-PEG₅₀₀₀-MAL (MW: 5546) and peptide (sequence: GSWKYWFGEGGC (SEQ ID NO: 2), MW: 1376.5) by a coupling reaction, in which a terminal cysteine on the peptide formed a thioether bond with the carboxyl group of maleimide on DSPE-PEG₅₀₀₀-MAL (FIG. 12).

DSPE-PEG₅₀₀₀-MAL (100 mg) and peptide (25 mg) having an equal molar ratio were dissolved in deionized water. The reaction mixture was gently stirred with a magnetic stirrer at 1,000 rpm at room temperature for 24 hours. After the reaction, the DSPE-PEG₅₀₀₀-peptide conjugate was characterized by matrix-assisted laser desorption ionization time of flight mass spectrometry (MALDI-TOF MS). In order to investigate the stability of DSPE-PEG₅₀₀₀-peptide in the processing condition of sonication, the freshly made peptide conjugate solution was processed with the sonication horn for 15, 30, 45, and 60 minutes at room temperature. Then they were subjected to MALDI-TOF MS to assess the degree of stability.

### Example 2.3. Preparation of R Encapsulated Nanocarriers

A mixture composed of the following lipids in weight were dissolved in methanol: 4 mg of R, 7 mg of soy PC, 22 mg of K, 22 mg of TA, and (5 mol % of PC). After mixing, methanol was removed using a nitrogen evaporator. After suspending Rnano lipid mixture in 76° C. deionized water, the suspension was homogenized for 1 minute followed by sonication for an additional 1 minute and placement on ice immediately thereafter.

ASC-targeted L-Rnano were made by replacing DSPE-PEG₅₀₀₀ with DSPE-PEG₅₀₀₀-peptide at an equal molar amount. After sonication, Rnano and L-Rnano were placed on ice immediately. Void nanocarriers (Vnano) and ligand-incorporated Vnano (L-Vnano) were prepared using the above materials and procedures without adding R. For in vitro binding and uptake experiments, fluorescence dye Rhoda (replacing 1 mol % of total PC) was added to make Rhoda-labeled nanocarriers. For in vivo imaging and flow cytometer experiments, near-infrared fluorescent dye DiD (replacing 1 mol % of total PC) was added to make DiD-labeled nanocarriers.

### Example 2.4. Characteristics, Encapsulation Efficiency, Loading Capacity of Nanocarriers

The particle size and polydispersity index (PI) and zeta potential values were measured using a Brookhaven BI-MAS particle size analyzer, and the zeta potential was measured using a Zeta PALS analyzer (Brookhaven Corporation, NY). The morphology and size of nanocarriers were determined using a 200 kV Hitachi H-8100 transmission electron microscope (TEM) instrument (Tokyo, Japan). The encapsulation efficiency and loading capacity of nanocarriers were measured as follows:

Encapsulation efficiency (%)=(Weight of R added−Weight of free R)/Weight of R added×100%

Loading capacity (%)=(Weight of R added−Weight of free R)/Weight of R-NPs×100%

### Example 2.5. Nanocarriers Physical Stability and In Vitro Release Study

To determine the stability of nanocarriers at different temperatures, the freshly made Rnano and L-Rnano were aliquoted into black tubes and stored at 4° C., 2° C., and 37° C. for 3 days. The mean particle size, PI and zeta potential were measured every day. The in vitro release behavior was measured in the dissolution medium, which was composed of 1× phosphate buffer saline (1×PBS, pH 7.4) and methanol (80:20, v/v), using a dialysis method. Free form of R, Rnano and L-Rnano containing 0.5 mg of R were dissolved and dispersed in 1 mL of dissolution medium and then placed in three different dialysis bags with MWCO 6,000-8,000. R released into the medium at each time point was determined using the Shimadzu high-performance liquid chromatography (HPLC) system (Shimadzu Corporation, Kyoto, Japan).

### Example 2.6. In Vitro Binding to and Uptake of Nanocarriers by ΔDCN Cells

ΔDCN cells were grown in Dulbecco's modified Eagle's medium (DMEM) containing 10% calf serum, 1% antibiotics (penicillin-streptomycin) and 5 μg/mL puromycin in a 5% CO₂, 37° C. environment.

### Example 2.7. Measurement of Binding and Uptake of Nanocarriers by Fluorescence Microscope

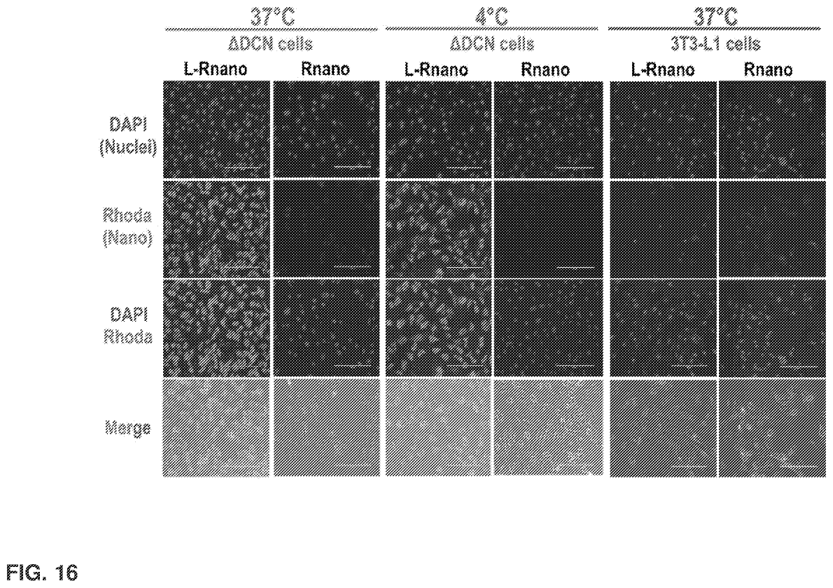

Before treating cells, Applicants measured incorporated Rhoda amounts in both non-targeted and ASC-targeted nanocarriers using a BioTek Microplate Reader and diluted them to ensure that both contained equal Rhoda amounts. ΔDCN cells (1×10⁵ cells/well) were cultured in a 24-well plate overnight and treated with Rhoda-labeled void nanocarriers or R encapsulated nanocarriers at either 4° C. or 37° C. for 2 hours. Cells were then washed three times with ice-cold 1×PBS and fixed with 3.7% formaldehyde in 1×PBS for 15 minutes at room temperature, followed by additional washing cells with ice-cold 1×PBS three times. After staining nuclei with 4′, 6-diamidine-2′-phenylindole dihydrochloride (DAPI) at room temperature for 10 minutes, cells were mounted and visualized under the EVOS® auto fluorescence microscope. Rhoda-labeled nanocarriers (λ_{exc}: 560 nm, λₑₘ: 583 nm) and cell nuclei stained by DAPI (λ_{exc}: 358 nm, λₑₘ: 461 nm) are shown in FIG. 16. To equal comparison of all images, the setting of the microscope was identical for all measures.

### Example 2.8. Measurement of Binding and Uptake of Nanocarriers by Flow Cytometry

Before treating cells, Applicants measured incorporated DiD (λ_{exc}: 644 nm, λₑₘ: 663 nm) amounts in both DiD-labeled non-targeted and ASC-targeted nanocarriers by using an IVIS system, and diluted them to ensure that both contained equal DiD amounts. Attached ΔDCN cells were trypsinized, resuspended in a centrifuge tube at a density of 1×10⁵ cells/mL and treated with DiD-labeled Rnano (DiD-Rnano) or DiD-labeled L-Rnano (DiD-L-Rnano). After incubating cells in dark at 37° C. for 2 hours, cells were centrifuged at 500×g for 5 minutes at 4° C. The supernatant was then removed.

Cells were then washed by resuspending them in an equal volume of 1 mL 1×PBS followed by centrifugation at 500×g for 5 minutes and removal of the supernatant. Cells were resuspended to the previous volume with flow buffer (1×PBS containing 1% bovine serum albumin) and were subjected to an Attune N×T Flow Cytometer. The binding and uptake of nanocarriers were determined by the co-localization rate of DiD fluorescence and ΔDCN cells.

### Example 2.9. Cellular R Content

ΔDCN cells (1×10⁵ cells/well) were cultured in a 6-well plate overnight and treated with the free form of R, Rnano and L-Rnano at either 4° C. or 37° C. for 4 hours. Cells were then washed three times with ice-cold 1×PBS and collected in 0.6 M acetic acid in a glass tube. Cellular R was extracted by ethyl acetate and determined by the high-performance liquid chromatography (HPLC) (Shimadzu instruments, Columbia, Md., USA). Briefly, ethyl acetate and quercetin (Q, internal standard) were added into the cells suspension along with mixing, sonication, and centrifugation. Supernatant ethyl acetate was dried using a nitrogen evaporator, resuspended in 100 μL methanol and injected into the HPLC system. The protein precipitates were dried, and then digested using 0.5 N NaOH. After overnight incubation, the bicinchoninic Acid (BCA) Kit was used for protein determination. Cellular R content was showed as μg of R per mg of cellular protein.

### Example 2.10. Ex Vitro Binding to and Uptake of Nanocarriers by the C57BL/6J Mouse Primary Stromal Vascular Fraction (SVF)

Sterile techniques, instruments and solutions were used in this experiment. Both inguinal WAT (I-WAT) and gonadal WAT (G-WAT) excised from C57BL/6J mice were washed with ice-cold 1×PBS. The WAT depots were weighted and rinsed in an isolation buffer (20 mM NaCl, 0.5 mM KCl, 1.2 mM KH₂PO₄, 0.6 mM MgSO₄.7H₂O and 0.9 mM CaCl₂.6H₂O, 20 mM HEPES, and 2.5% BSA). WAT depots were then minced with a scissor and a blade and added to the isolation buffer in a ratio of 1 g of WAT to 3 mL of isolation buffer, which was supplemented with Type 1 collagenase at a concentration of 280 U/mL. Minced WAT was digested in a shaking water bath at 200 rpm for 45 minutes at 37° C. Digested WAT was filtered through 100 μm nylon mesh (Spectrum, Rancho Dominquez, Calif.) to get a single cell suspension. After centrifugation at 500×g for 5 minutes at 4° C., floating mature adipocytes were removed and the pellets of the stromal fraction were collected and washed twice with the isolation buffer. After counting, cells were plated into 6-well plates with growth media [DMEM containing 10% fetal bovine serum (FBS, Atlas biological, CO), 1% antibiotics (penicillin/streptomycin)] in a humidified incubator at 37° C. and 5% CO₂. After 24 hours, unattached cells were removed by extensive washing with 1×PBS. The attached cells were maintained in the same medium, which was changed every other day until they reached 70-80% confluence.

For measurement of binding and uptake of nanocarriers using fluorescence imaging, incorporated Rhoda amounts in both Rhoda-labeled Rnano and L-Rnano (Rhoda-Rnano and Rhoda-L-Rnano) were measured using a BioTek Microplate Reader. Mouse primary SVF (1×10⁵ cells/well) was cultured in a 24-well plate reach to 80% confluence and treated with Rhoda-Rnano and Rhoda-L-Rnano at either 4° C. or 37° C. for 2 hours. Then, cells were washed, fixed, nuclei stained, mounted and visualized under the EVOS® Auto fluorescence microscope.

To measure R content in mouse primary SVF, primary SVF cells (3×10⁵ cells/well) were cultured in 6-well plates until they reached 80% confluence. The cells were then treated with 10 μM of the free form of R, Rnano and L-Rnano at either 4° C. or 37° C. for 4 hours. Cells were then washed three times with ice-cold 1×PBS and collected in 0.6 M acetic acid in a glass tube. Cellular R was extracted using ethyl acetate and measured by the HPLC system.

### Example 2.11. ASC Target Specificity of Nanocarriers in C57BL/6J Mice

Male 6-week old C57BL/6J purchased from the Jackson Laboratory were fed a high-fat diet (HFD) (45% energy from fat, D12451, Research Diets, Inc, New Brunswick, N.J.) for 4 weeks. Mice were housed at 22° C. to 24° C., 45% relative humidity, and a daily 12/12 light/dark cycle. They drank and ate the HFD ad libitum. Body weights of mice at the time of experiments were around 30 g. Before injection, Applicants measured incorporated DiD amounts in both DiD-labeled non-targeted Rnano and ASC-targeted L-Rnano using an IVIS system and diluted them to ensure that they contained equal DiD amounts. Based on the body weight, C57BL/6J mice were grouped, and received DiD-labeled non-targeted or ASC-targeted nanocarriers (DiD-Vnano or DiD-L-Vnano; DiD-Rnano or DiD-L-Rnano) containing equal amounts of DiD via tail vein injection. The animal protocol was approved by the animal care and use committee of Texas Tech University, Lubbock, Tex.

### Example 2.12. In Vivo Targeting of Nanocarriers to WAT

After 24 hours of post-injection, mice were imaged using the IVIS system. Mice were then sacrificed and perfused with 1×PBS through the left ventricle of the heart. The fluorescence reflectance images of the dissected liver, BAT, retroperitoneal WAT (RP-WAT), I-WAT and G-WAT were visualized using the IVIS system.

### Example 2.13. ASC Target Specificity of Nanocarriers

After imaging the WAT depots (I-WAT and G-WAT) using the IVIS system, the SVF of each WAT depot was enzymatically digested and resuspended in flow buffer as described above. Floating mature adipocytes were collected, washed twice with flow buffer and kept on ice. The SVF cells were washed, lysed by 1×RBC lysis buffer, counted and resuspended in flow buffer at 1×10⁶ cells/100 μL. Then, the SVF cells were stained with the following fluorophore-conjugated antibodies with optimal dilution: PE anti-mouse CD34 antibody (λ_{exc}: 480 nm, λₑₘ: 575 nm), Brilliant Violet 421™ anti-mouse CD31 antibody (λ_{exc}: 405 nm, λₑₘ: 421 nm), Brilliant Violet 421™ anti-mouse CD45 antibody (λ_{exc}: 405 nm, λₑₘ: 421 nm) and Alexa Fluor® 594 anti-mouse CD29 antibody (λ_{exc}: 590 nm, λₑₘ: 617 nm) (Biolegend; San Diego, Calif., USA) on ice for 30 minutes and protected from light. The cells that stained with CD31 (endothelial cells marker) and CD45 (hematopoietic cells marker), which were considered as negative controls. The cells were pelleted and washed twice with flow buffer. Then, FACS analysis was performed using an Attune N×T flow cytometer.

### Example 2.14. Animal Studies

Sixty male 6-week old C57BL/6J mice from Jackson's lab were fed with a high-fat diet (HFD) (45% energy from fat, Research Diets, Inc, D12451) for 9 weeks starting from week 1 (age 7 weeks old) to week 9 (age 15 weeks old). The mice were allowed to drink and eat the HFD ad libitum and maintained at 22-24° C., 45% relative humidity and a daily 12/12 hour light/dark cycle. After 4 weeks, mice were weighed and randomly assigned into one of the following six treatment groups: treatment 1: saline; treatment 2: Vnano; treatment 3: L-Vnano; treatment 4: free R (15 mg/kg body weight/day); treatment 5: Rnano (15 mg/kg body weight/day); and treatment 6: L-Rnano (15 mg/kg body weight/day). Treatments were intravenously injected into mice via tail veins twice a week. Food intake and body weight were recorded weekly. Glucose tolerance test (GTT) was conducted at week 8 and the insulin tolerance test (ITT) and cold tolerance test were conducted at week 9.

After 9 weeks, mice were fasted overnight and humanely sacrificed. Blood was collected from the abdominal vein and brain, liver, lung, spleen, kidneys, skeletal muscle, gonadal white adipose tissue (G-WAT), inguinal WAT (I-WAT), retroperitoneal WAT (RP-WAT), and BAT of each mouse. Each tissue was cut into 3 pieces to be immediately frozen in liquid nitrogen followed by storage at −80° C. and fixed in 4% paraformaldehyde (for histology).

### Example 2.15. Body Composition

Body composition of mice was performed at week 0, 2, 4 and 5 of treatments using an EchoMRI™ Whole Body Composition Analyzer (MRI system) (EchoMRI LLC, Houston, Tex., USA).

### Example 2.16. Glucose and Insulin Tolerance Tests Assays

GTT and ITT were performed at end of treatments to assess whole-body glucose and insulin tolerance. For GTT, mice were fasted for 6 hours and then injected intraperitoneally with glucose at a dose of 1 mg/kg body weight. Blood was collected and blood glucose concentrations were measured with a One Touch® glucometer from tail vein blood at 0, 15, 30, 60, 90, and 120 minutes post-injection. To measure ITT, mice were fasted for 4 hours and then injected intraperitoneally with insulin at a dose of 0.75 U/kg body weight. Blood glucose concentrations were measured as above at 0, 15, 30, 45, and 60 minutes post-injection. The area under the curve (AUC) was calculated. Blood glucose concentrations were plotted against time and the area under curve was calculated by following formula:

AUC=((C₁+C₂)/2)×(T₂−T₁)

C: Glucose concentration (mg/dL)

T: Time (minute)

### Example 2.17. Cold Tolerance Test

Initial rectal temperature was measured before placing the mice into a cold room (4±1° C.). Rectal temperature was measured using a thermometer, which was inserted 1 cm into the rectum. The rectal probe was wiped with alcohol swabs (70% isopropyl alcohol) and coated with mineral oil or glycerol between each measurement. Mice (5 mice/treatment) were housed individually in a cage without bedding in the cold room. Mice had free access to food and water. Mice were kept in the cold room for 6 hours and the rectal temperature was measured hourly.

### Example 2.18. Measurement of Plasma Lipid Profile, Insulin and Glucose Concentrations

After animal sacrifice, fasting blood was collected and plasma was obtained by centrifugation at 3,000 rpm at 4° C. for 25 minutes. Plasma concentrations of triglyceride (TG), total cholesterol (TC), high-density lipoprotein cholesterol (HDL-C), low-density lipoprotein cholesterol (LDL-C), and very low-density lipoprotein cholesterol (VLDL-C) were measured at the Jean Mayer USDA human nutrition research center on aging at Tufts university, Boston, Mass., USA.

Fasting plasma glucose concentrations were measured by One Touch® glucometer. Fasting insulin concentrations were determined by an insulin Millipore® ELISA commercial kit (Millipore, Billerica, Mass., USA). The insulin resistance was evaluated using homeostatic model assessment of insulin resistance (HOMA-IR) by the following formula:

HOMA-IR=[Fasting plasma glucose (mg/dL)×Fasting plasma insulin (mU/L)]×(405)⁻¹

### Example 2.19. Measurement of WAT R Content

I-WAT and G-WAT (around 100 mg) were homogenized in 1 mL of saline (0.9% sodium chloride) with 10 μL of 0.1 mg/mL of quercetin as an internal standard. After vortexting for 1 minute, 1 mL of ethyl acetate was added into the mixture. After vortexing, the above mixture followed by centrifugation at 10,000 rpm and 4° C. for 10 minutes, the upper aqueous phase was transferred into a new tube. Another 1 mL of ethyl acetate was added to the bottom phase to repeat extraction. The combined ethyl acetate was dried under nitrogen gas. The dried R was reconstituted by methanol and subsequently placed in a seal vial for high-performance liquid chromatography (HPLC) analysis (Shimadzu instruments, Columbia, Md., USA) and determined using a reversed-phase column C18. The mobile phase was composed of water and methanol (50/50, v/v) containing 1% acetic acid. Ten microlitres of the sample solution were injected into the chromatograph, and the analysis was performed at room temperature. Detection was routinely accomplished by monitoring the absorbance signals at 310 nm.

### Example 2.20. Measurement of Liver R Content

Liver (around 100 mg) was homogenized in 1 mL of saline. β-glucuronidase from Helix pomatia (Type H-3, Sigma, St. Louis, Mo.) and sulfatase from Helix pomatia (Type H-1, Sigma, St. Louis, Mo.) were then added. After vortexting, the mixture was incubated at 37° C. for 2 hours to convert R derivatives to native R. To the aforementioned samples were added 10 μL of 0.1 mg/mL of quercetin as internal standard and 1 mL of ethyl acetate. The samples were then processed in the vortex for 1 minute prior to centrifugation at 10,000 rpm at 4° C. for 10 minutes. The supernatant was placed into a new tube. The residue was extracted one more time with 1 mL of ethyl acetate, followed by centrifugation. The combined ethyl acetate of the supernatants was evaporated under nitrogen gas. The dried R was reconstituted by methanol and subsequently measured by a HPLC system as described above.

### Example 2.21. Real-Time Polymerase Chain Reaction (PCR)

Total RNA was extracted from I-WAT, G-WAT, and BAT using a TRIzol reagent (ThermoFisher Scientific, Waltham, Mass., USA). cDNA was synthesized from quantified RNA using a Maximal First Strand cDNA Synthesis Kit (Thermo Scientific, Pittsburgh, Pa., USA) according to the manufacturer's instructions. cDNA levels of target genes were measured using power SYBR green master mix (Applied Biosystems, Austin, Tex.) on a Real-time PCR system (Eppendorf Mastercycler® ep realplex instrument, NY). The mRNA-fold change was calculated using the 2^{-ΔΔCt} method, which normalizes against housekeeping gene 36B4. Primer sequences of target genes are listed in Table 1.

TABLE 1
Primer sequences

| | | | |
|---|---|---|---|
| 36B4 | Forward | SEQ ID NO: 3 | GCTTCGTGTTC |
| | | | ACCAAGGAGGA |
| | Reverse | SEQ ID NO: 4 | GTCCTAGACCA |
| | | | GTGTTCTGAGC |
| UCP-1 | Forward | SEQ ID NO: 5 | GCTTTGCCTCA |
| | | | CTCAGGATTGG |
| | Reverse | SEQ ID NO: 6 | CCAATGAACAC |
| | | | TGCCACACCTC |
| PGC-1α | Forward | SEQ ID NO: 7 | GAATCAAGCCA |
| | | | CTACAGACACC |
| | | | G |
| | Reverse | SEQ ID NO: 8 | CATCCCTCTTG |
| | | | AGCCTTTCGTG |
| PPAR-γ | Forward | SEQ ID NO: 9 | GTACTGTCGGT |
| | | | TTCAGAAGTGC |
| | | | C |
| | Reverse | SEQ ID NO: 10 | ATCTCCGCCAA |
| | | | CAGCTTCTCCT |
| PRDM16 | Forward | SEQ ID NO: 11 | ATCCACAGCAC |
| | | | GGTGAAGCCAT |
| | Reverse | SEQ ID NO: 12 | ACATCTGCCCA |
| | | | CAGTCCTTGCA |
| CD137 | Forward | SEQ ID NO: 13 | CGTGCAGAACT |
| | | | CCTGTGATAAC |
| | Reverse | SEQ ID NO: 14 | GTCCACCTATG |
| | | | CTGGAGAAGG |
| Tmem26 | Forward | SEQ ID NO: 15 | ACCCTGTCATC |
| | | | CCACAGAG |
| | Reverse | SEQ ID NO: 16 | TGTTTGGTGGA |
| | | | GTCCTAAGGTC |
| Leptin | Forward | SEQ ID NO: 17 | TGGGGTTTTGG |
| | | | AGCAGTTTG |
| | Reverse | SEQ ID NO: 18 | CTGTCACTCTT |
| | | | TCCCGGTCT |
| Adiponectin | Forward | SEQ ID NO: 19 | AGGCCGTTCTC |
| | | | TTCACCTAC |
| | Reverse | SEQ ID NO: 20 | ACTTCCAGACA |
| | | | GTCATGCGA |
| MCP-1 | Forward | SEQ ID NO: 21 | TCGCTCAGCCA |
| | | | GATGCAAT |
| | Reverse | SEQ ID NO: 22 | ATCTCCTTGGC |
| | | | CACAATGGTC |

### Example 2.22. Immunohistochemistry Staining

The aforementioned formalin-fixed I-WAT was embedded in paraffin, and cut to 5 μm sections by the Department of Pathology of TTU Health Sciences Center. Deparaffinized and rehydrated sections were incubated with the anti-UCP-1 antibody (U6382, Sigma, St. Louis, Mo.) for overnight at 4° C., followed by incubation with biotinylated secondary antibody for 1 hour. Then the sections were developed utilizing avidin-conjugated horseradish peroxidase (HRP) with diaminiobenzidine (DAB) as substrate by using a Vectastain ABC kit (Vector Laboratories, Burlingame, Calif., USA) according to the manufacturer's instructions. Following development, the slides were mounted under coverslips with Permount and images were taken under an Evos XL core microscope (AMG, Bothell, Wash., USA) after 24 hours of drying.

### Example 2.23. Hematoxylin and Eosin (H&E) Staining

H&E staining of I-WAT was conducted by the Department of Pathology of Texas Tech University Health Sciences Center. Briefly, the paraffin-embedded I-WAT sections (5 μm) were deparaffinized and rehydrated with xylene and ethanol. Sections were cleaned with water to skim reagent residue. Excess water was then blotted, the sections were incubated with Hematoxylin for 4 minutes, and washed several times using water. The sections were stained with Eosin and dehydrated. Finally, the sections were cleaned and covered with xylene-based mounting medium.

### Example 2.24. Detection of Inflammation-Related Cytokines in Plasma

Cytokine measurement (TNF-α, MCP-1, IL-6, IFN-γ and IL-10) in the plasma were detected using a bead-based LEGENDplex™ Mouse Inflammation Panel (BioLegend, San Diego, Calif.) according to the manufacturer's instructions and using an Attune N×T flow cytometer. The data were analyzed using LEGENDplex™ analysis software.

### Example 2.25. Histological Examination

After 5 weeks of treatment, 5 mice from each treatment group were randomly selected for safety evaluation. After terminal exsanguinations under isoflurane, the heart, liver, lungs, kidneys, skeletal muscle, brain, and spleen of each mouse were collected, measured, weighed and described in detail. They were fixed, embedded, sectioned, and stained for histological examination and evaluation, which was conducted by pathologists from the Texas veterinary medical diagnostic laboratory (TVMDL), College Station, Tex., USA.

### Example 2.26. Statistical Analysis

Data analysis was performed using Statistical software “R”. One-way ANOVA followed by Tukey HSD test was performed to compare multiple groups. Differences were considered statistically significant at p<0.05. Data in figures and tables are expressed as means±standard error of the mean (SEM).

### Example 2.27. Results: The Development of ASC-Targeted R Loaded Nanoparticles

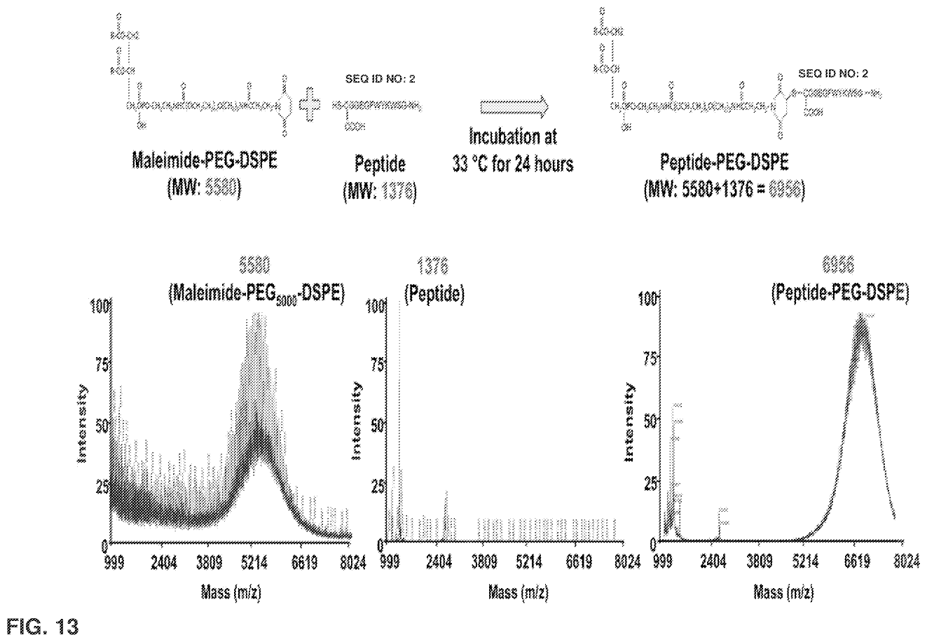

To exploit the peptide as ligand incorporated on the surface for the assembly of ASC-targeted nanoparticles, Applicants first synthesized DSPE-PEG₅₀₀₀-peptide from DSPE-PEG₅₀₀₀-maleimide and peptide by a coupling reaction, in which a terminal cysteine on the peptide formed a thioether bond with the carboxyl group of maleimide. The conjugation was confirmed by MALDI-TOF (FIG. 13).

The MALDI-TOF spectra of production after conjugating reaction exhibited the average mass of DSPE-PEG₅₀₀₀-peptide at m/z 6963 (FIG. 13), while that of peptide showed a single and sharp peak at m/z 1376 (FIG. 13). The difference in the masses of DSPE-PEG₅₀₀₀-peptide and peptide was 5580 Da, which corresponded to the mass of DSPE-PEG₅₀₀₀-maleimide (FIG. 13) and indicated the successful conjugation.

Next, the ligand incorporated R loaded nanoparticles (L-Rnano) were synthesized by using soy PC, Kolliphor® HS15 and αTA, for which DSPE-PEG₅₀₀₀-peptide can be loaded on the lipid surface. Many different formulae have been investigated to achieve desired nanoparticle size and encapsulation efficiency to utilize as a drug delivery system.

The spherical shape of L-Rnano, characterized by transmission electron microscope (TEM), was largely similar to Rnano and the change. The surface texture and the enlargement of the size of L-Rnano may due to the distribution of DSPE-PEG₅₀₀₀-peptide (FIG. 13). The polydispersity index (PDI) of freshly made Rnano and L-Rnano were 0.315 and 0.341, respectively and the zeta potentials of Rnano and L-Rnano were −19 mV and −10 mV, indicating that DSPE-PEG₅₀₀₀-peptide incorporation did affect the surface charge of nanoparticles. Furthermore, both Rnano and L-Rnano showed an encapsulation efficiency of 95.8±0.2% and 96.2±0.4%, respectively. Nevertheless, the loading capacity of L-Rnano (22.3±0.6%) was lower than the Rnano (29.2±0.8%), which may due to high molecular weight DSPE-PEG₅₀₀₀-peptide incorporated.

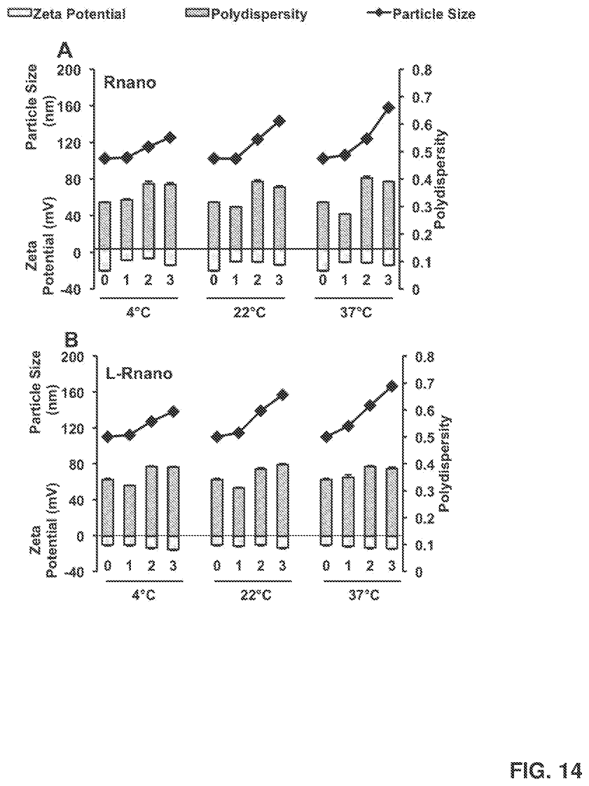

As a desired factor for the therapeutic potential of blood circulation and drug delivery, the physical stability of Rnano and L-Rnano, in terms of their particle size, PDI and zeta potentials were monitored over time in the presence of saline at 4° C., 22° C., and 37° C. As shown in FIG. 14, the particles size of Rnano and L-Rnano did not change at 4° C. and 22° C. for 24 hours and increased by 17.6% and 18.8% at 4° C., by 28.6% and 27% at 22° C. following three days of storage, respectively.

The PI of Rnano and L-Rnano were slightly increased around 15% at 4° C. and 22° C. after 3 days of storage. After incubating nanocarriers at 37° C. for 3 days, the particle size of Rnano and L-Rnano were increased by 35.4% and 33.7%, respectively. Under the same conditions, the PI values of Rnano and L-Rnano were increased by 19.2% and 11%, respectively. The above results indicated the optimal physical stability of Rnano and L-Rnano at 37° C. Further, the zeta potential of Rnano and L-Rnano did not change significantly at the above temperatures over three days.

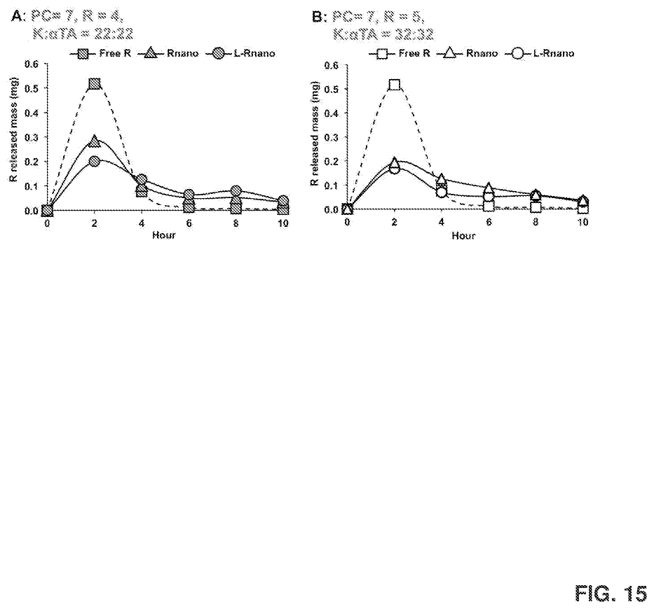

A dialysis method was applied for the determination of in vitro release pattern of R from either Rnano or L-Rnano. In this study, Applicants compared R release mass and percentage of accumulative released R in between free R, Rnano and L-Rnano (FIG. 15). In the first two hours, only 0.05 mg R released from the dialysis bag containing Rnano and LRnano, which is around 2% of total R loaded by nanoparticles. In contrast, more than 0.13 mg of R released from the native R dialysis bag during the same time, which is around 35% of total free R. In the following six hours, the mass of R released from free R bag was lower than 0.02 mg at every time point and undetected after eight hours, while for Rnano and L-Rnano, R was continuously released and the percentage of accumulative R release of Rnano and LRnano has no significant difference. These results suggested that Rnano and L-Rnano had a sustained R release property.

### Example 2.28. Targeting and Browning Effect of L-Rnano In Vitro

Applicants studied the binding affinity and cellular uptake of L-Rnano by ΔDCN-transduced 3T3-L1 cells (ΔDCN cells) and isolated mouse primary stromal vascular cells (SVCs). Rnano and L-Rnano loaded with a fluorescent dye, Rodamine (Roda), were incubated with either ΔDCN cells for 2 h at 4° C. and 37° C. (FIG. 16) or 3T3-L1 cells at 37° C. (FIG. 16). Fluorescent microscopy images revealed that the internalization of the Roda-labeled L-Rnano (red color) was significant in ΔDCN cells at both 4° C. and 37° C., but was rarely observed in 3T3-L1 cells, which lacked the ΔDCN receptor. The results demonstrate the specific role of ΔDCN receptor on the surface of ΔDCN cells for the uptake of targeted nanoparticles.

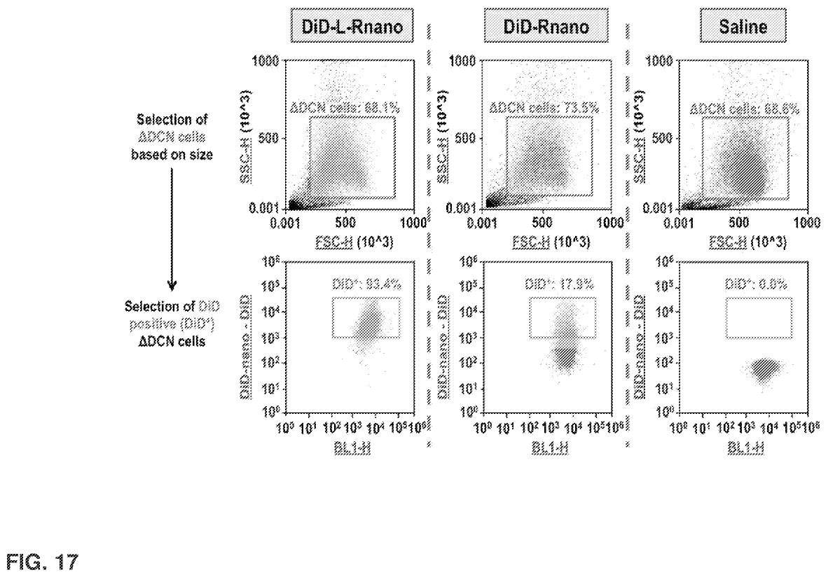

To confirm the aforementioned results, Applicants used flow cytometry to measure the percentage of ΔDCN cells containing DiD fluorescence signals to compare the targeting specificity of DiD-labeled Rnano and L-Rnano in vitro (FIG. 17). When assessed by ligand, the DiD positive rate of ΔDCN cells, treated with DiD-labeled L-Rnano, was enhanced five-fold more efficiently than DiD-labeled Rnano.

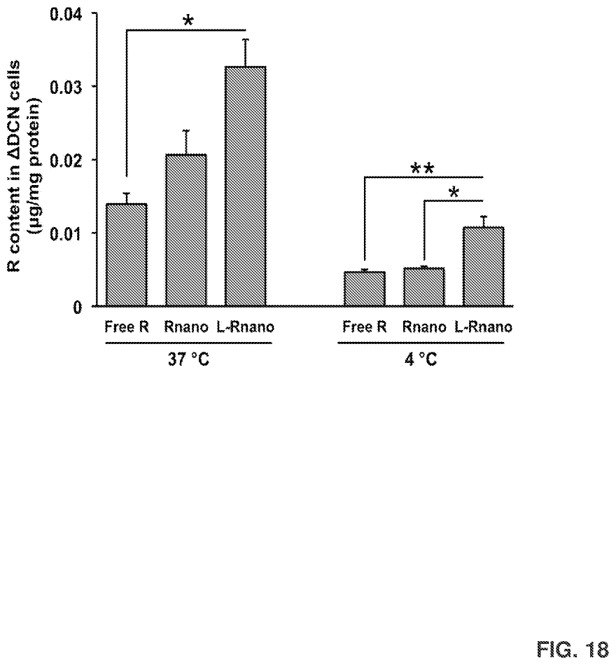

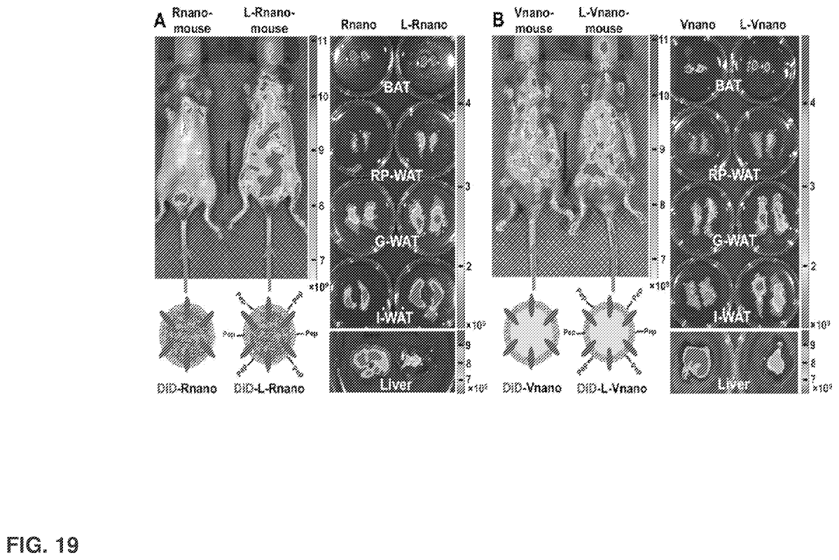

Next, to investigate the targeted L-Rnano uptake by ΔDCN cells, ΔDCN cells were treated with free R, Rnano or L-Rnano at both 4° C. and 37° C. for 4 hours (FIG. 18). Consistent with above observation, targeted L-Rnano increased ΔDCN cells R content two-fold higher when compared to free R and Rnano at 4° C. because of its high targeting specificity to ΔDCN receptors. Also, compared to free R, the ΔDCN cells treated with both Rnano and L-Rnano increased cellular R content 0.67 and 1.46 fold, respectively at 37° C.

Next, to identify the cell-specificity of binding and cellular uptake of targeted L-Rnano to ASC, which has endogenous surface ΔDCN receptors, Applicants treated equal amounts of Roda-labeled Rnano and L-Rnano with I-WAT SVCs isolated from C57BL/6J mice. Consistent with ΔDCN cells binding images above, Rhoda-L-Rnano compared to Rnano had higher binding effect to SVCs at both 37° C. and 4° C. Applicants further validated cellular uptake of free R, Rnano and L-Rnano of SVCs by measuring cellular content of R upon each treatments. L-Rnano-treated SVCs had 2.5-fold (p=0.02) and 2.8-fold (p=0.07) more cellular R content than free R-treated SVF cells at 37° C. and 4° C., respectively. Based on above data, Applicants concluded that the nanoparticles carried with DSPE-PEG₅₀₀₀-peptide ligand were rapidly targeted to ΔDCN receptors expressed cells and were efficiently uptaken into the cells.

### Example 2.29. ASC-Targeting Specificity and Anti-Obesity Effects of L-Rnano in C57BL/6J Mice

Before demonstrating the in vivo therapeutic anti-obesity efficacy, the DSPE-PEG₅₀₀₀-peptide carried nanoparticles were examined for WAT-ASC-targeting in C57BL/6J mice by IVIS® Spectrum in vivo imaging system (IVIS) and fluorescence-activated cell sorting system. To compare the Rnano and L-Rnano WAT targeting performance, DiD-labeled Rnano and L-Rnano were intravenously injected into C57BL/6J mice, followed by monitoring the fluorescence biodistribution of whole body and harvested organs and fat depots upon necropsy 24 h after injection through IVIS. The fluorescent signals of the subcutaneous and intraperitoneal fat area of the whole body of mouse treated with DiD-labeled L-Rnano were enhanced in comparison with that of the DiD-labeled Rnano treated mouse. As shown in FIG. 19, no significant difference was observed between the biofluorescence intensity of BAT in DiD-labeled Rnano and L-Rnano group. In addition, the fluorescent intensity in the WAT (RP-WAT, G-WAT and I-WAT) for the DiD-L-Rnano mouse was higher than that in WAT for the DiD-Rnano mouse, indicating that DiD-L-Rnano did accumulate within WAT after injection, especially in I-WAT. Especially, DiD-labeled L-Rnano exhibits higher inhibitory effect against liver uptake and accumulation due to the ASC-targeting capacity of L-Rnano.

For a better understanding of the targeting efficiency of L-Rnano to WAT-derived ASC, Applicants performed flow cytometry to investigate the level of colocalization of DiD-labeled nanoparticles and ASC isolated by fluorescence-activated cell sorting (FACS) from SVF cell suspensions from RP-WAT, G-WAT and I-WAT. After the imaging of fat pads, the SVF was isolated from each WAT fat pad and prepared for the following FACS gating and analyzing.

The size of SVF populations is typically smaller than 20 μm, and this feature makes it possible to separate SVF from the cell debris during initial FSC versus SSC gating. To characterize the ASC in SVF, Applicants further employed antibody combinations for flow cytometric analysis. As surface marker of endothelial and hematopoietic cells, CD31⁻ and CD45⁻ had been used as the gate for the identification of ASC from SVF. In addition, CD31⁻CD45⁻ population had been gated by CD34 and CD29, which were two mesenchymal cell markers that were expressed on the surface of ASC. As expected, the ASC containing DiD signal increases in the WAT isolated from DiD-L-Rnano-injected mouse due to the incorporation of ASC-targeting peptide.

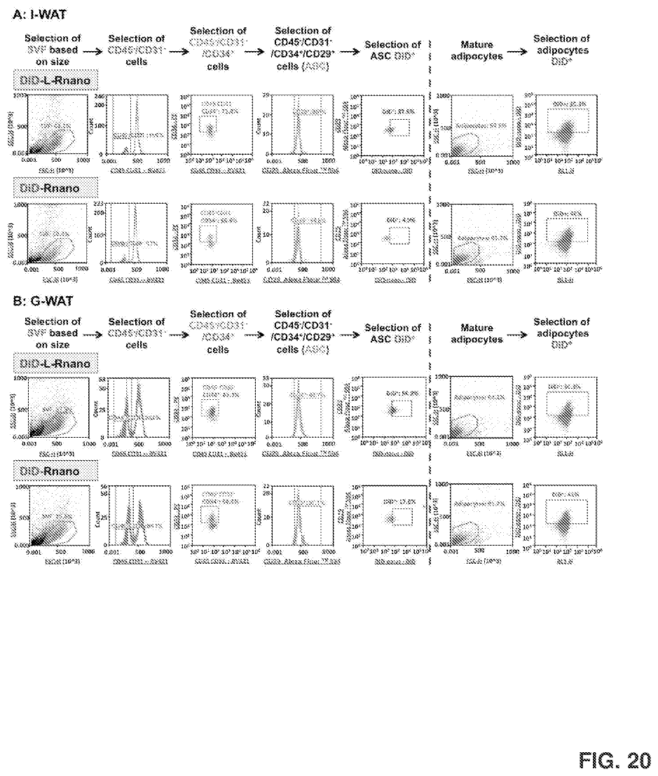

According to the flow cytometric measurement, the percentages of DiD⁺ ASC in visceral WAT (RP and G-WAT) of DiD-L-Rnano mouse were 4.8 and 3.5-fold higher than that of the DiD-Rnano mouse, respectively (FIG. 20). Interestingly, there was a 9.3-fold higher DiD⁺ ASC in the DiD-L-Rnano mouse, as compared to DiD-Rnano treatment, achieving 31.7% cell ratio among the ASC population (FIG. 20). Furthermore, the dot plot of mature white adipocytes isolated from WAT treated with either DiD-labeled L-Rnano or Rnano had been presented as well, and no differences were found from the percentage of DiD containing mature white adipocytes, collected from each WAT depot and treatment (FIG. 20).

The aforementioned results demonstrate that the peptide, identified as the ASC-targeting ligand, incorporated on nanoparticles, has a high binding affinity to ΔDCN-expressing ASC in vivo, especially the I-WAT derived ASC. This WAT-ASC-specific targeting is desirable for the anti-obesity effects, as a targeting delivery system of R to enhance accumulation in ASC and further induce the differentiation of ASC to beige adipocytes.

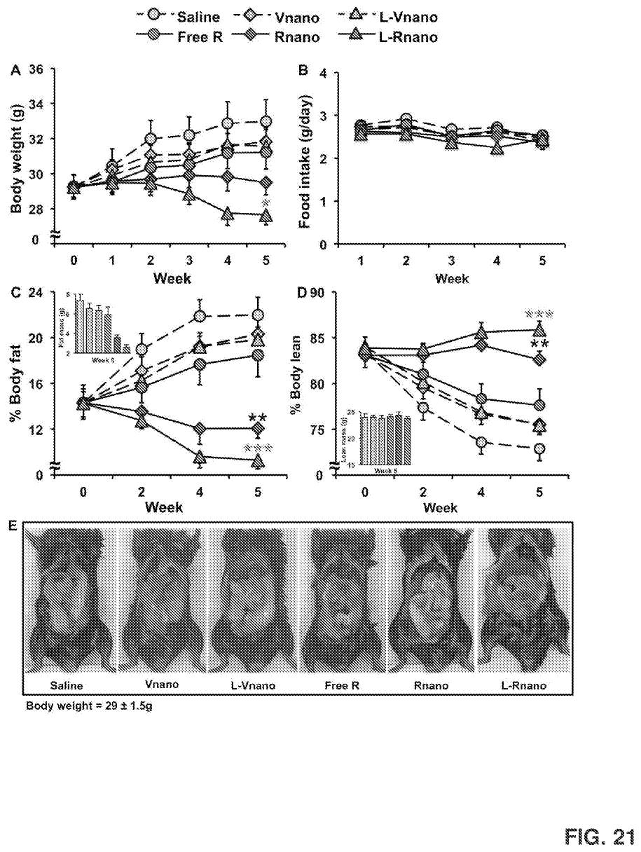

To investigate the anti-obesity effects and metabolic benefits of L-Rnano, Applicants administrated free from of R (free R), Rnano and L-Rnano to C57BL6J mice maintained on a high-fat diet. In order to validate the benefits generated by R, saline, void particles (Vnano) and ligand-Vnano (L-Vnano) were taken as a control for this experiment. As compared to saline, treatment of L-Rnano for 5 weeks significantly lowered body weight, reflected in the reduction of percentage of body fat (% body fat) (FIG. 21).

The food intake activity and lean mass were unaffected by L-Rnano treatment. Therefore, the effects on body weight and % body fat was due to an increase in energy expenditure. Free R and Rnano-treated mice had a slightly lower % body fat than the saline-treated mice, while the changes of % body fat were similar among saline, Vnano and L-Vnano groups (FIG. 21).

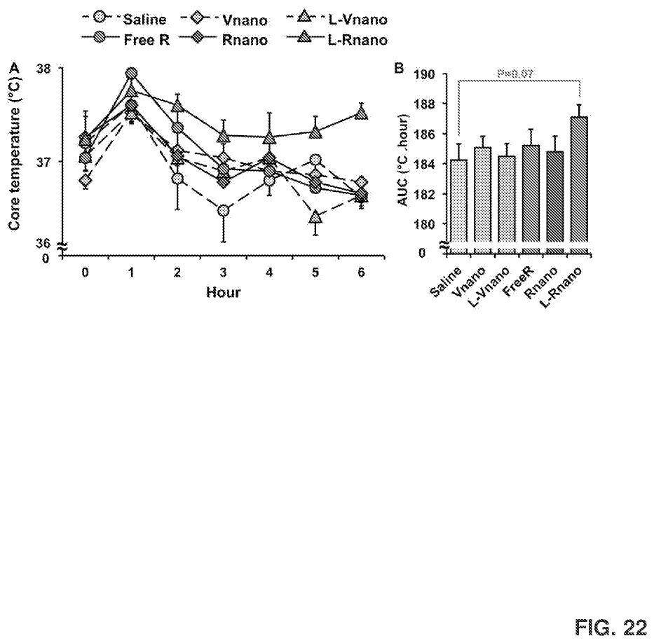

To further validate the enhancement of energy expenditure of mice treated by L-Rnano, Applicants conducted a cold tolerance test and recorded mice rectal body temperature changes for 6 hours. There were no significantly differences in the basal core temperature of mice (0 hour) among 6 treatment groups. However, the body temperature maintaining ability of L-Rnano-treated mice was improved remarkably during the acute cold challenge, with higher rectal body temperature at almost every time point as compared to other treatment groups of mice (FIG. 22). At hour 6, L-Rnano-treated compared to saline-treated mice had a 0.8±0.06° C. higher rectal temperature.

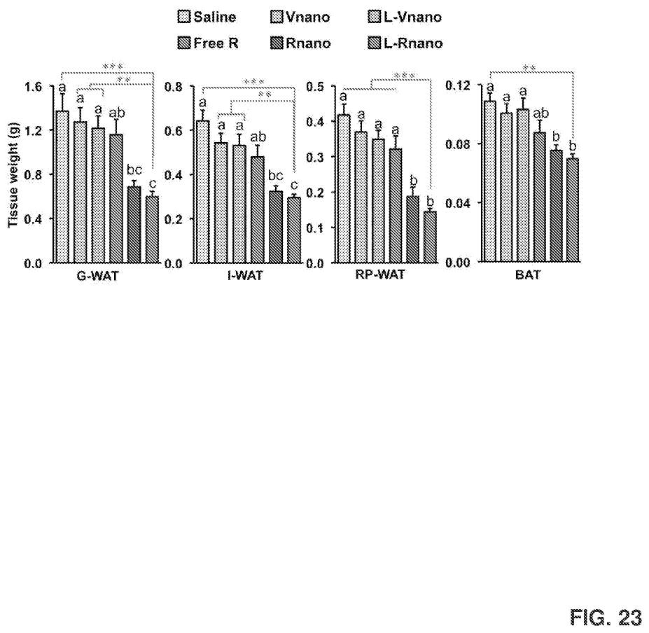

Next, after mice excision, Applicants performed further studies by measuring and analyzing the weights of fat depots and the size of adipocytes. Mice treated with Rnano and L-Rnano showed significantly decreased weights of G-WAT, I-WAT, RP-WAT, and BAT, and reduced lipid deposition, which were consistent with the reduction of body weight and % body fat (FIG. 23). These alterations were associated with decreased size of adipocytes.

There were smaller lipid droplets in the I-WAT of Rnano and L-Rnano-treated mice, with the diameter of adipocytes lower than 30 μm and 20 μm, respectively, while diameter of most adipocytes in saline, Vnano, L-Vnano and free R groups were in the diameter around 100 μm. Interestingly, adipocytes from L-Rnano-treated mice showed the multilocular lipid droplet morphology, a general characteristic of brown/beige adipocytes, suggesting that L-Rnano induced browning of I-WAT. Applicants further quantified mean areas of I-WAT adipocytes by counting area/cell in randomly selected microscopic fields and found that the area of cells of L-Rnano mice significantly lowered than that of saline mice (FIG. 23). The aforementioned results indicate that L-Rnano exerted anti-obesity effects by reducing % body fat and the adipocyte size in mice.

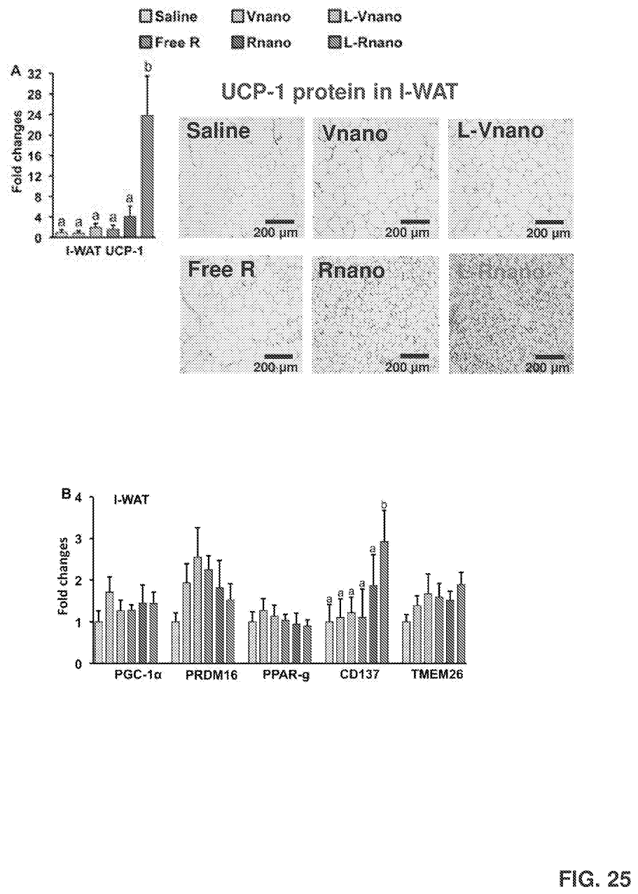

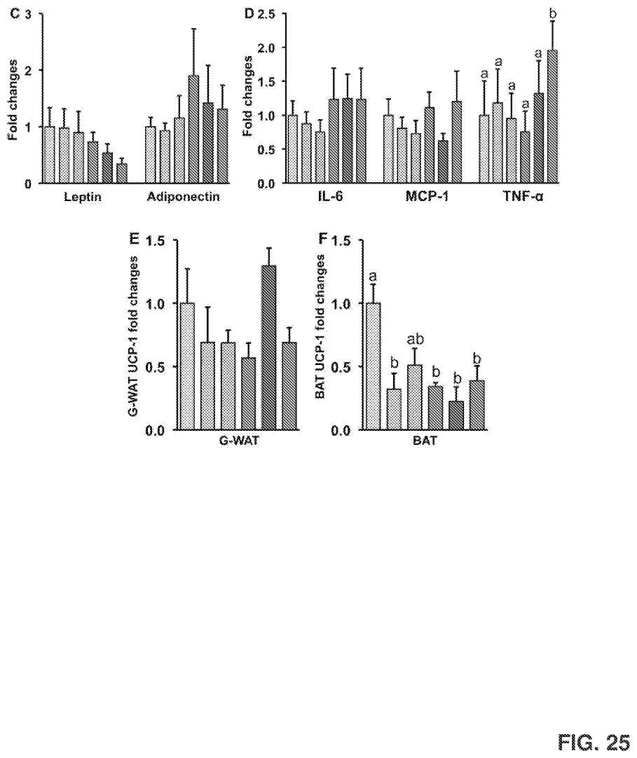

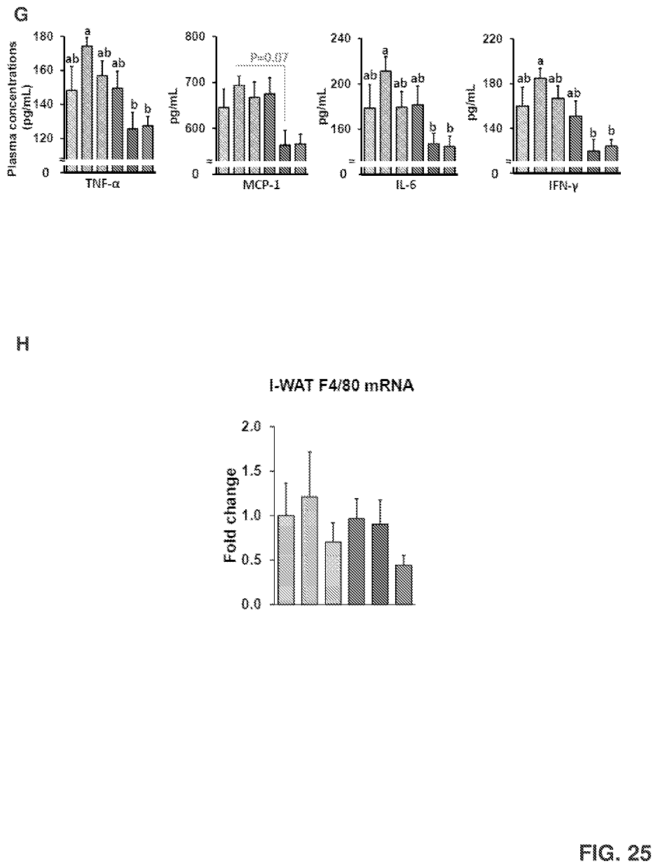

Next, Applicants compared the thermogenic function of I-WAT between L-Rnano-treated mice and all of the other control mice by measuring the mRNA and protein levels of UCP-1 (FIG. 25). As compared to saline, Rnano slightly increased the UCP-1 mRNA expression in I-WAT, but no significance was found, whereas, L-Rnano-treated mice had a significantly increased 20-fold (p=0.001) than to that for saline mice (FIG. 25).

In agreement, the UCP-1 protein stained by immunohistochemistry (IHC) in L-Rnano-treated mice had the highest UCP-1 staining and levels in I-WAT among 6 treatment groups. Applicants further examined whether the mRNA levels of other thermogenic genes were activated in the I-WAT by L-Rnano and found that L-Rnano significantly increased the gene expression of CD137 (p=0.03), which serves as a classic beige marker (FIG. 25). However, there were no differences in expression of PGC-1α, PRDM16, and PPAR-γ among all treatment groups.

Previously, Applicants found the significantly enhanced accumulation of L-Rnano in ASC by flow cytometry. Here, Applicants measured the R content in I-WAT, G-WAT and liver. As compared to free R and Rnano-treated mice, L-Rnano-treated mice had a significant 4-fold and 3-fold higher R accumulation in I-WAT, respectively, and there was no significant difference in I-WAT R content between free R and Rnano mice, suggesting that ASC-targeting ligand coated nanoparticle system enhanced high WAT-derived ASC target specificity, which resulted in enhanced therapeutic browning and weight loss efficacy. Conversely, L-Rnano-treated mice had significantly decreased R accumulation in liver than that of mice treated with free R, consistent with Applicants' observation of IVIS fluorescence results.

### Example 2.30. Health Effects of L-Rnano in C57BL/6J Mice

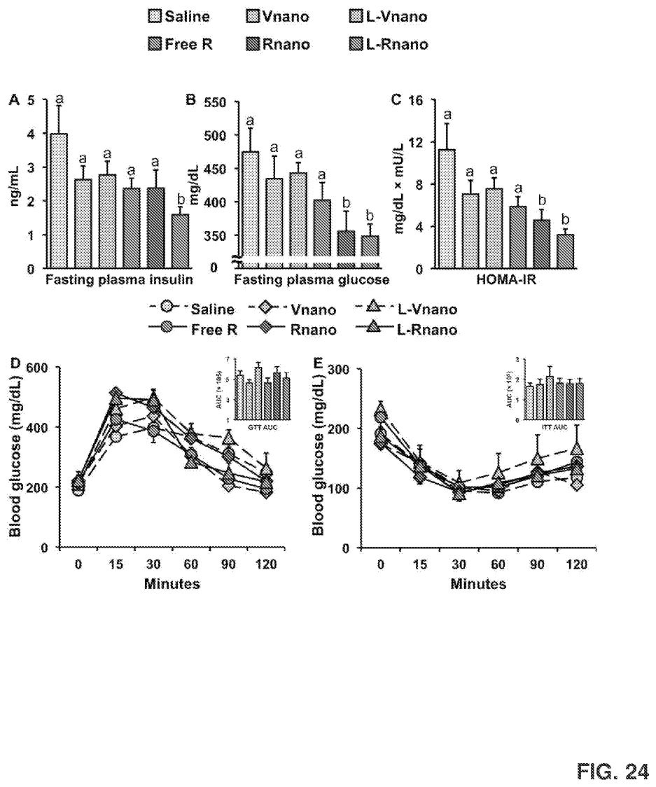

The increase in I-WAT thermogenic effect of mice suggested that L-Rnano may protect the mice from HFD-induced insulin resistance. Therefore, Applicants analyzed the fasting plasma glucose and insulin concentrations and calculated the HOMA-IR, which was often used to assess insulin resistance. All forms of R treatments (free R, Rnano, and L-Rnano) reduced fasting blood insulin and glucose concentrations, as compared to the saline group (FIG. 24).

In particular, L-Rnano when compared to saline significantly reduced fasting blood insulin and glucose concentrations by 60% and 26%, respectively (FIG. 24).

Applicants also found a significant reduction in HOMA-IR in L-Rnano-treated mice, indicating that insulin resistance was prevented by the treatment of L-Rnano (FIG. 24). In relation to this, leptin plasma level showed a significant reduction by L-Rnano as well, suggesting the improvement of leptin resistance (FIG. 24). Applicants further determined the effects of L-Rnano on the gene expression of leptin in I-WAT and found that L-Rnano-treated mice had the lowest leptin mRNA expression, which was paralleled with its lowest fat pad weight (FIG. 24).

Inflammatory factors known to be produced and secreted by WAT were found to be elevated in obesity (FIG. 25). Applicants investigated the relationship between pro-inflammatory markers in circulation and L-Rnano's WAT browning effects by measuring plasma concentration of different cytokines (FIG. 25). Applicants found that TNF-α, IL-6, IFN-γ and MCP-1 concentrations in plasma were significantly lowered in Rnano and L-Rnano-treated mice than that of saline mice (FIG. 25).

These reductions may be due to an improvement of macrophage infiltration in WAT. Thus, Applicants measured F4/80 mRNA level in I-WAT to determine if macrophages were affected by L-Rnano treatment. Five weeks after L-Rnano administration, a 2-fold lower amount of F4/80 mRNA level in I-WAT was observed when compared mice injected with saline (FIG. 25).

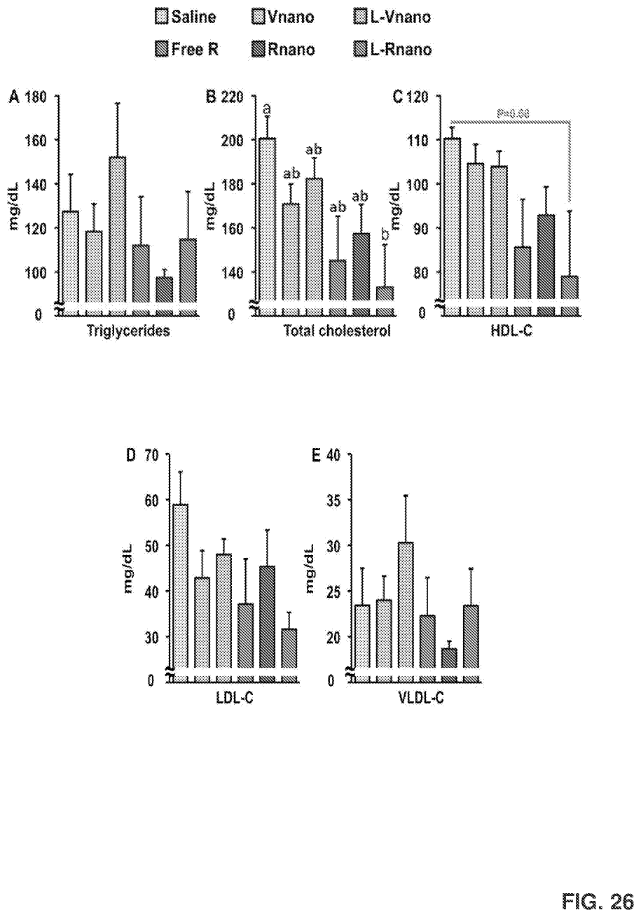

Circulation of lipid in bold systems was associated with the risk of cardiovascular disease. Total triglyceride, cholesterol, HDL-C, LDL-C and VLDL-C were examined in the plasma. Applicants found that L-Rnano-treated compared to saline mice had significant reductions in blood concentrations of TC and reduction of HDL-C and LDL-C (FIG. 26). More significantly, I-WAT clears 33% of the total cholesterol from the circulation (FIG. 26). In addition, beige adipocytes are sufficient to alter energy expenditure and lipid profile.

### Example 2.31. Safety Evaluation

In order to evaluate the biosafety of WAT-derived ASC-targeted nanoparticles delivery system, Applicants used histological analysis formalin-fixed paraffin-embedded organs to investigate the systemic toxic side effects on mice of 5 weeks intravenous (IV) injection of each treatment. In heart and aorta samples, adipose tissue adjacent to the aorta infiltrated by a moderate number of lymphocytes was observed in saline-treated mice. Rare foamy macrophages were found in alveolar spaces of lung in a few mice. Additionally, analysis of the liver sections revealed that a small number of hepatocytes were expanded in cytoplasms of a few mice of 6 treatment groups.

The aforementioned histopathologic findings in those mice were considered incidental and can be commonly observed in mice. No significant findings were observed in brain, spleen, kidney and skeletal muscle of all treatments. The aforementioned results suggest no organ damage or lesion occurred after nanoparticle delivery applications.

### Example 2.31. Discussion of Experimental Results

Although the effects of R on the formation of beige adipocytes in WAT have been investigated in animal studies, the clinical applications of R as an anti-obesity supplement have been hindered by its extremely low aqueous solubility, poor bioavailability and indiscriminate distribution in the human body. In this Example, the utilization of nanoparticles has overcome the aforementioned issues by increasing R's aqueous solubility and chemical stability.

However, simple enhancement of R's bioavailability cannot consequently ensure the accumulation of R into the WAT and further delivery of R into the WAT-derived ASCs. To increase the ASC-targeting specificity of nanoparticles in the body, Applicants synthesized the nanoparticles with ligand to form ASC-targeting nanoparticles R delivery system to achieve more effective anti-obesogenic therapy.

In the present nanoparticles system, soy PC and DSPE were used to form the monolayer of nanoparticle membrane, which provided biodegradable characteristics. The hydrophilic heads of soy PC and DSPE faced the outward aqueous environment and two hydrophobic fatty acid tails buried the vitamin E acetate core and thereby encapsulated R into the core, which consequently protected R from degradation during blood circulation. The 100 nm size in diameter of both Rnano and L-Rnano allowed the particles to penetrate into adipose tissues easily and be eliminated by the liver and other organ systems slowly.

In addition, the PEG₅₀₀₀ on the surface of nanoparticles can prolong the circulation of nanoparticles by stabilizing them against opsonization. To examine whether ASC-targeted L-Rnano can effectively target to ΔDCN receptor specifically, Applicants used ΔDCN cells and primary mouse SVF as an in vitro model and C57BL/6J mice as an in vivo model.

Applicants' in vitro fluorescence cells images indicated that L-Rnano accumulation was associated with the receptor-dependent binding effect because the fluorescence enhancement of L-Rnano is specifically observed for ΔDCN cells, but not for 3T3-L1 cells, confirming the functional interaction between ASC-targeting ligand and ΔDCN receptor. Additionally, compared with the free form of R and non-targeted Rnano, L-Rnano showed very effective ΔDCN cellular uptake especially at 37° C., indicating that receptor-mediated endocytosis is energy-dependent and is an efficient and target-specific mechanism to internalize nanoparticles into the cells.

As an additional validation of L-Rnano binding ΔDCN receptor in vitro, Applicants demonstrated its enhanced accumulation to primary WAT-derived SVF, followed by effective cellular uptake. L-Rnano did not bind SVF cells as well as ΔDCN cells, which may due to the presence of endothelial cells and other heterogeneous population of SVF cells, and disappearance of ΔDCN protein on the ASC surface caused by overnight culturing of SVF.

In agreement with in vitro binding data, to further investigate the targeting interaction between ligand incorporated L-Rnano and endogenous ΔDCN receptors of WAT ASCs, L-Rnano's biodistribution in WAT in vivo and internalization of ASCs were conducted using HFD-induced C57BL/6J mouse. The difference in fluorescent intensities between WAT and liver were observed for Rnano and L-Rnano groups, indicating that ligand enabled the nanoparticles to bypass the liver and enhance accumulation in the WAT due to the following reasons. Initially, DSPE-PEG₅₀₀₀-peptide, instead of DSPE-PEG₅₀₀₀, provided higher PEG polymer density on the surface of L-Rnano to slow the hepatic clearance due to the polymer's hydrophobic block. The brush-like conformation created by DSPE-PEG₅₀₀₀-peptide effectively reduced the hepatic deposition. Moreover, the peptide Applicants applied in this targeted nanoparticles delivery system was screened for ASC homing specifically, which may help L-Rnano bypass the liver. Noticeably, this high in vivo ASC targeted efficacy of the ligand-coated L-Rnano, validated by fluorescent biodistribution and FASC, showed promising potential as a drug-delivery nanoparticles system to ASCs for anti-obesity therapy and enabling their targeted accumulation.

Although ASCs have many clinical potentials based on their capacity for proliferation and differentiation, very rare therapies targeted at ASCs were present until the ΔDCN receptor was identified as a validated molecular target specific for these cells. In this Example, Applicants investigated the anti-obesity effects of this innovative ASC-targeted L-Rnano on the formation of beige adipocytes in WAT and other health beneficial effects induced by this process. It has been reported that R concentrations used in published animal studies to inhibit white adipogenesis, stimulate adipocytes lipolysis, induce beige and brown adipocytes activation and other beneficial metabolic effects were in the general range from 0.04% to 0.4% contained in diet (w/w). Previous studies indicated that 0.1% R (equal to 110 mg R/kg body weight/day, 30 g mouse) can induce the beige adipogenesis in mouse I-WAT and the brown adipocyte formation in mouse interscapular BAT.

In order to protect the nanoparticle structure and ligand function from degradation in the gut, Applicants decided to deliver treatments via IV injection. According to the bioavailability of R in animals and humans, R concentration (15 mg R/kg BW/day) was selected to be used in this Example.

In agreement with previous studies, Applicants found that free R, Rnano and L-Rnano decrease the body weight gain compared with saline group in this intervention animal study. After a 4 week obesogenic diet challenge, mice were obese and significant WAT composed body mass. To induce WAT browning, a relative large dose of R is required to be delivered in the tissue. As a conventional drug delivery approach, free R leads to off-target R accumulation in other organs, such as the liver. Therefore, no significant reduction of body weight, % body fat and adipocytes size can be observed in free R-treated mice due to the low amount of R accumulation in WAT.

Applicants' results also validated that nano-encapsulation of R improved its aqueous solubility and bioavailability in blood circulation, since the body weight and % body fat of Rnano-treated mice were decreased significantly. More importantly, ASC-targeted L-Rnano significantly reversed mice's obese condition, which is contributed by the ligand incorporated system that specifically delivered R to WAT, facilitated R entry into the target ASCs to induce thermogenic WAT browning, and then further minimized the R accumulation in liver and off-target adverse effects.

Applicants' observation also found that, L-Rnano compared to saline significantly upregulated I-WAT UCP-1 mRNA expression by approximately 20-fold, and further confirmed by UCP-1 IHC staining, which was paralleled with the R content in I-WAT. In addition, the expression levels of selective markers of the beige adipocytes, such as CD137 and TMEM26 in the L-Rnano-treated group was much higher than those of the saline group.

Compared to the highly susceptible browning potential of I-WAT, G-WAT was quite resistant to browning, although compared to the other two forms of R, G-WAT in mice treated with L-Rnano have higher R content, which suggested that the mechanisms and potentials involved in increasing beige adipocytes formation in WAT in different anatomical locations throughout the animal body might be different.

The aforementioned observations clearly indicated that ASC-targeted therapy inducing WAT, especially I-WAT, into browning was a highly promising strategy for effective obesity intervention.

Overall, this Example provided a proof of the anti-obesity therapeutic potential of targeted L-Rnano, which induced formation of thermogenic beige adipocytes in I-WAT, with profound impact on health benefits, such as insulin resistance, inflammation and blood lipid. Previous studies indicated that I-WAT browning improved insulin sensitivity. However, in this Example, L-Rnano only lowered HOMA-IR values, which indicated greater insulin sensitivity, but no affect on GTT and ITT.

Although GTT remained the most commonly performed test to examine glucose tolerance, the fact was that the GTT measured not only insulin sensitivity but also glucose effectiveness. Therefore, more specific insulin sensitivity measurement, for example, the hyperinsulinemic euglycemic glucose clamp technique, which has been described as the gold standard method for determining insulin sensitivity, is required to perform in the future study. To further explain the insulin data, Applicants measured pro-inflammatory cytokine levels in blood, since obesity was associated with a chronic low-grade systemic inflammation. Applicants found that, paralleled with body weight reduction, both Rnano and L-Rnano significantly decreased the plasma levels of TNF-α and IL-6, which were the inflammatory cytokines produced mainly by WAT and linked with insulin resistance, glucose tolerance and blood TG elevation. This reduction may be due to an improvement of macrophage infiltration in WAT, as suggested by the decrease in MCP-1, a critical chemokine for macrophage recruitment. Other cytokines synthesized within WAT have been measured, such as Th cytokines IFN-γ and IL-10. IL-10 served as an anti-inflammatory cytokine, generated by M2 macrophages and protected adipocytes from TNF-α-induced insulin resistance. Unfortunately, the increase of IL-10 in blood was not observed in mice treated with either Rnano or L-Rnano.

Recent studies indicated that BAT has the ability to enhance TG-rich lipoproteins clearance from the circulation. More importantly, BAT has been identified as a key player in cholesterol metabolism by accelerating the hepatic clearance of the cholesterol-enriched remnants and promoting HDL reverse cholesterol transport. However, whether beige adipocytes have the similar TG and cholesterol clearance effects is unclear. Applicants found that L-Rnano significantly lowered plasma TC, HDL-C and LDL-C concentrations, suggesting that the induction of WAT browning could enhance the selective uptake of FA derived from lipolysis of TRLs by beige adipocytes, and subsequently accelerate the hepatic clearance of the cholesterol-enriched remnants. However, L-Rnano had no effects on plasma TG clearance. Therefore, a longer-term of L-Rnano treatment may be required to observe the clearance phenomenon.

In summary, Applicants' study demonstrated that it is possible to specifically deliver R to ASCs in expanded WAT using targeted nanoparticles system in vivo to induce beige adipocytes formation in I-WAT and consequently treat obesity. Therefore, Applicants envision that the nanoparticles can be utilized as effect anti-obesity therapies for human obesity.

The results in this Example also indicate that, in addition to the inhibition of obesity, an effective browning effort induced by L-Rnano in I-WAT contributed to improved metabolic health. Moreover, the ASC-targeted nanoparticles drug delivery system can carry a broad range of functional agents or modulators that could facilitate ACS-based biomedical and translational studies and minimize off-target adverse effects.

Additional experimental results can be found in an article by Zu et al. entitled “Resveratrol liposomes and lipid nanocarriers: Comparison of characteristics and inducing browning of white adipocytes.” Colloids and Surfaces B: Biointerfaces 164 (2018) 414-423.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method for delivering one or more active agents to adipose stromal cells through the use of a delivery agent, said method comprising:
associating the adipose stromal cells with the delivery agent, wherein the delivery agent comprises:
a particle,
one or more active agents carried by the particle, and
a targeting agent associated with the particle,
wherein the targeting agent directs the delivery agent to the adipose stromal cells; and
wherein the associating results in the delivery of the one or more active agents into the adipose stromal cells.

2. The method of claim 1, wherein the particle is a lipid-based particle comprising a phospholipid.

3. (canceled)

4. The method of claim 1, wherein the particle lacks triglycerides.

5. The method of claim 1, wherein the particle contains triglycerides.

6. The method of claim 1, wherein the particle further comprises an active agent stabilizer or an excipient,
wherein active agent stabilizer or excipient is co-incorporated with the one or more active agents within the particle, and
wherein the active agent stabilizer or excipient is selected from the group consisting of an antioxidant, vitamin E, vitamin C, vitamin A, triglyceride, uric acid, glutathione, triglycerides, monosaccharides, disaccharides, polysaccharides, fibers, lipids, vitamins, minerals, phytochemicals, proteins, terpenoids, or combinations thereof.

7-11. (canceled)

12. The method of claim 1, wherein the particle further comprises a surfactant on a surface of the particle.

13. (canceled)

14. The method of claim 1, wherein the particle comprises a surface with a negative charge.

15. The method of claim 1, wherein the particle is in the form of nanoparticles, wherein the nanoparticles comprise diameters ranging from about 20 nm to about 200 nm.

16. (canceled)

17. The method of claim 1,
wherein the particle comprises a hydrophobic core, and
wherein the one or more active agents comprise hydrophobic active agents that are within the hydrophobic core.

18-19. (canceled)

20. The method of claim 1, wherein the one or more active agents are dispersed within the particle in the form of an amorphous phase.

21. The method of claim 1, wherein the one or more active agents are selected from the group consisting of small molecules, peptides, polypeptides, proteins, hydrophobic active agents, hydrophilic active agents, drugs, nucleotides, RNA, shRNA, siRNA, miRNA, DNA, nutrients, phytochemicals, and combinations thereof.

22. The method of claim 1, wherein the one or more active agents have a concentration of more than 1 nM or more than 1 LM.

23. (canceled)

24. The method of claim 1, wherein the one or more active agents comprise resveratrol.

25. The method of claim 1, wherein the one or more active agents are encapsulated within the particle.

26. The method of claim 1, wherein the targeting agent is selected from the group consisting of amino acids, peptides, proteins, aptamers, antibodies, small molecules, carbohydrates, polysaccharides, lipids, and combinations thereof.

27. The method of claim 1, wherein the targeting agent is associated with en a surface of the particle through a linker, wherein the linker is covalently coupled to a surface of the particle and to the targeting agent.

28. (canceled)

29. The method of claim 27, wherein the linker comprises polyethylene glycol.

30. The method of claim 1,
wherein the targeting agent targets an epitope on the adipose stromal cells,
wherein the epitope is a receptor on adipose stromal cells, and
wherein the delivery of the one or more active agents into the adipose stromal cells occurs by receptor-mediated endocytosis.

31. The method of claim 30, wherein the epitope is a cleavage product of decorin

32. (canceled)

33. The method of claim 1, wherein the targeting agent comprises a peptide selected from the group consisting of CSWKYWFGEC (WAT 7) (SEQ ID NO: 1), GSWKYWFGEGGC (SEQ ID NO: 2), and combinations thereof.

34. (canceled)

35. The method of claim 1,
wherein the adipose stromal cells are selected from the group consisting of adipose stromal stem cells, adipose stromal progenitor cells, and combinations thereof, and
wherein the adipose stromal cells are a component of a white adipose tissue, a brown adipose tissue, a beige adipose tissue, and combinations thereof.

36. (canceled)

37. The method of claim 1, wherein the associating occurs in vitro.

38. The method of claim 1,
wherein the associating occurs in vivo in a subject,
wherein the associating comprises administering the delivery agent to the subject,
wherein the delivery agent is used to treat or prevent a disorder or disease in the subject, and
wherein the disorder or disease is selected from the group consisting of metabolic syndromes, diabetes, type 2 diabetes, cardiovascular diseases, hypertension, coronary heart diseases, insulin resistance, dyslipidemia, cancer, osteoarthritis, rheumatoid arthritis, aging, wrinkles, alopecia, liver failure, multiple sclerosis, obesity, and combinations thereof.

39-40. (canceled)

41. The method of claim 38,
wherein the delivery agent is used to treat or prevent obesity in the subject, and
wherein the delivery agent treats or prevents obesity by conversion of white adipose tissue to brown adipose tissue, beige adipose tissue, brown-like adipose tissue, or combinations thereof in the subject.

42. (canceled)

43. The method of claim 1, wherein the delivery agent is embedded within hydrogels.

44-81. (canceled)


SEQUENCE LISTING (22 sequences)

SEQ ID NO: 1 - protein, 10 aa, Artificial Sequence
  Peptide-Based Targeting Agent (WAT 7)
CSWKYWFGEC

SEQ ID NO: 2 - protein, 12 aa, Artificial Sequence
  Peptide-Based Targeting Agent
GSWKYWFGEGGC

SEQ ID NO: 3 - protein, 22 aa, Artificial Sequence
  36B4 Forward Primer
GCTTCGTGTTCACCAAGGAGGA

SEQ ID NO: 4 - protein, 22 aa, Artificial Sequence
  36B4 Reverse Primer
GTCCTAGACCAGTGTTCTGAGC

SEQ ID NO: 5 - protein, 22 aa, Artificial Sequence
  UCP-1 Forward Primer
GCTTTGCCTCACTCAGGATTGG

SEQ ID NO: 6 - protein, 22 aa, Artificial Sequence
  UCP-1 Reverse Primer
CCAATGAACACTGCCACACCTC

SEQ ID NO: 7 - protein, 23 aa, Artificial Sequence
  PGC-1(alpha) Forward Primer
GAATCAAGCCACTACAGACACCG

SEQ ID NO: 8 - protein, 22 aa, Artificial Sequence
  PGC-1(alpha) Reverse Primer
CATCCCTCTTGAGCCTTTCGTG

SEQ ID NO: 9 - protein, 23 aa, Artificial Sequence
  PPAR-(gamma) Forward Primer
GTACTGTCGGTTTCAGAAGTGCC

SEQ ID NO: 10 - protein, 22 aa, Artificial Sequence
  PPAR-(gamma) Reverse Primer
ATCTCCGCCAACAGCTTCTCCT

SEQ ID NO: 11 - protein, 22 aa, Artificial Sequence
  PRDM16 Forward Primer
ATCCACAGCACGGTGAAGCCAT

SEQ ID NO: 12 - protein, 22 aa, Artificial Sequence
  PRDM16 Reverse Primer
ACATCTGCCCACAGTCCTTGCA

SEQ ID NO: 13 - protein, 22 aa, Artificial Sequence
  CD137 Forward Primer
CGTGCAGAACTCCTGTGATAAC

SEQ ID NO: 14 - protein, 21 aa, Artificial Sequence
  CD137 Reverse Primer
GTCCACCTATGCTGGAGAAGG

SEQ ID NO: 15 - protein, 19 aa, Artificial Sequence
  Tmem26 Forward Primer
ACCCTGTCATCCCACAGAG

SEQ ID NO: 16 - protein, 22 aa, Artificial Sequence
  Tmem26 Reverse Primer
TGTTTGGTGGAGTCCTAAGGTC

SEQ ID NO: 17 - protein, 20 aa, Artificial Sequence
  Leptin Forward Primer
TGGGGTTTTGGAGCAGTTTG

SEQ ID NO: 18 - protein, 20 aa, Artificial Sequence
  Leptin Reverse Primer
CTGTCACTCTTTCCCGGTCT

SEQ ID NO: 19 - protein, 20 aa, Artificial Sequence
  Adiponectin Forward Primer
AGGCCGTTCTCTTCACCTAC

SEQ ID NO: 20 - protein, 20 aa, Artificial Sequence
  Adiponectin Reverse Primer
ACTTCCAGACAGTCATGCGA

SEQ ID NO: 21 - protein, 19 aa, Artificial Sequence
  MCP-1 Forward Primer
TCGCTCAGCCAGATGCAAT

SEQ ID NO: 22 - protein, 21 aa, Artificial Sequence
  MCP-1 Reverse Primer
ATCTCCTTGGCCACAATGGTC